(19) United States
(12) Patent Application Publication
(10) Pub. No.: US 2012/0077689 A1
(43) Pub. Date: Mar. 29, 2012

(54) Compartment-Specific Non-HLA Targets for Diagnosis and Prediction of Graft Outcome

(76) Inventors: Minnie M. Sarwal, Portola Valley, CA (US); Atul J. Butte, Stanford, CA (US)

(21) Appl. No.: 13/201,838

(22) PCT Filed: Feb. 16, 2010
(86) PCT No.: PCT/US10/00448

§ 371 Date: Dec. 12, 2011

Related U.S. Application Data
(60) Provisional application No. 61/207,939, filed on Feb. 17, 2009.

(51) Int. Cl. C40B 30/04; G01N 33/567; C12Q 1/68

(52) U.S. Cl. 506/9; 435/6.12; 435/7.21

(57) ABSTRACT

Methods and composition are provided for diagnosing or predicting the status or the outcome of a graft transplant. In some embodiments, the presence or absence of one or more proteins recognizing a non-HLA/non ABO antigen is determined. The obtained result is then employed to diagnose or predict the status or outcome of the graft transplant. Also provided are compositions, systems and kits that find use in practicing the subject methods.

### CROSS-REFERENCE

This application claims the benefit of U.S. Patent Application Ser. No. 61/207,939, filed on Feb. 17, 2009, which is herein incorporated by reference in its entirety.

### STATEMENT AS TO FEDERALLY SPONSORED RESEARCH

This invention was made with the support of the United States government under research grant numbers NIAID (R01 AI61739) and NLM (K22 LM008261) from National Institute of Allergy and Infectious Diseases and the National Institute of Health. The government has certain rights in this invention.

### BACKGROUND OF THE INVENTION

Despite advances in immunosuppressive therapies and the resultant reduction in the incidence of acute rejection, declining graft function remains a paramount clinical concern, as recent studies have shown no benefit of the reduction of acute rejection incidence on graft life expectancy (Pascual M et al. (2002) N Engl J Med 346, 580-590). This may partly relate to the heterogeneity of the acute rejection injury which may as yet be inadequately treated resulting in irreversible graft injury (Sarwal M, et al. (2003) N Engl J Med 349, 125-138; Kirk A D et al. (1997) Proc Natl Acad Sci USA 94, 8789-8794), but there is extensive evidence that antibodies recognizing and engaging with donor antigens, leading to humoral types of rejection, also play a key role in renal allograft outcomes (Terasaki P & Mizutani K (2006) Clin J Am Soc Nephrol 1, 400-403).

Antibodies recognizing HLA molecules (major histocompatibility antigens) are the most important and well known group of antibodies for renal transplantation. These HLA antibodies can be present prior to transplantation, due to prior exposure to non-self HLA molecules (after pregnancy, blood transfusion or prior allo-transplantation), or can be produced de novo after transplantation (Akalin E & Pascual M (2006) Clin J Am Soc Nephrol 1, 433-440). Donor-specific anti-HLA alloantibodies initiate rejection through complement-mediated and antibody-dependent, cell-mediated cytotoxicity (Vongwiwatana A, et al. (2003) Immunol Rev 196, 197-218). The accumulation of the complement degradation product C4d is generally regarded as a marker for an antibody-mediated alloresponse and is associated with poor graft survival (Mauiyyedi S, et al. (2002) J Am Soc Nephrol 13, 779-787).

In contrast to these ‘major’ histocompatibility antibodies, ‘minor’ non-HLA antigens have been implicated in renal allograft outcome, and likely have a much stronger role in clinical transplantation than previously thought (Opelz G (2005) Lancet 365, 1570-1576). Antibodies against MICA, a locus related to HLA determining a polymorphic series of antigens similar to HLA, have been associated with decreased graft survival (Terasaki P I et al. (2007) Am J Transplant 7, 408-415; Zou Y et al. (2007) N Engl J Med 357, 1293-1300). There is a suggestion that Duffy (a chemokine receptor, the Duffy antigen-receptor for chemokines [DARC]), and to a lesser extent Kidd polymorphic blood group antigens, are associated with chronic renal allograft histological injury (Lerut E et al. (2007) Transfusion 47, 28-40). In addition, antibodies against Agrin, the most abundant heparin sulfate proteoglycan present in the glomerular basal membrane, have been implicated in transplant glomerulopathy (Joosten S A et al. (2005) Am J Transplant 5, 383-393). Finally, agonistic antibodies against the Angiotensin II type 1 receptor (AT₁R-AA) were described in renal allograft recipients with severe vascular types of rejection and malignant hypertension (Dragun D et al. (2005) N Engl J Med 352, 558-569.).

It is expected that there are many more yet unidentified antigens that might evoke specific antibody responses after renal transplantation (Opelz G (2005) Lancet 365, 1570-1576). However, the identification of these non-HLA non-ABO immune targets is particularly difficult, and without target antigen identification, antibody screening for specificity is nearly impossible. The advent of high-density protein microarrays has made screening for serum antibodies against thousands of human proteins much more efficient. These protein arrays have been used successfully in auto-immune disease (Leitner W W et al. (2003) Nat Med 9, 33-39) and cancer (Hudson M E et al. (2007) Proc Natl Acad Sci USA 104, 17494-17499).

### SUMMARY OF THE INVENTION

Methods and composition are provided for diagnosing or predicting the status or the outcome of a graft transplant. In some embodiments, the presence or absence of one or more proteins recognizing a non-HLA/non ABO antigen is determined. The obtained result is then employed to diagnose or predict the status or outcome of the graft transplant. Also provided are compositions, systems and kits that find use in practicing the subject methods.

In some embodiments, the non-HLA/non ABO antigen is a graft-specific antigen. In some embodiments, the presence or absence of a plurality of proteins recognizing non-HLA/non ABO antigens is determined. In some embodiments, the expression of the non-HLA/non ABO antigens is also measured by methods such as PCR or microarrays. In some embodiments the protein recognizing a non-HLA/non ABO antigen is an antibody. In some embodiments the non-HLA/non ABO antigen is a graft-compartment specific antigen.

In another aspect, the invention provides methods for diagnosing or predicting graft status or outcome by determining the presence or absence of a plurality of antibodies recognizing non-HLA/non ABO antigens in a sample from a subject who has received a graft using a protein array. In some embodiments, the non-HLA/non ABO antigens are graft specific antigens. In some embodiments, the non-HLA/non ABO antigens are graft-compartment specific antigens.

In another aspect, the invention provides method for diagnosing or predicting graft status or outcome by determining the presence or absence of an antibody response against a graft compartment specific non-HLA/non ABO antigen in a sample from a subject who has received a graft. In some embodiments, the methods further comprises determining the presence or absence of a plurality of antibodies recognizing non-HLA/non ABO antigens, wherein the non-HLA/non ABO antigens are graft compartment specific antigens.

The graft status or outcome may comprise rejection, tolerance, non-rejection based graft injury, graft function, graft survival, chronic graft injury, or titer pharmacological immunosuppression. In some embodiments, the non-rejection based graft injury is selected from the group of ischemic injury, virus infection, peri-operative ischemia, reperfusion injury, hypertension, physiological stress, injuries due to reactive oxygen species and injuries caused by pharmaceutical agents.

In some embodiments, the sample from a subject who has received a graft is blood, serum, urine, or a stool sample

In some embodiments, the graft is selected from the group consisting of kidney graft, heart graft, liver graft, pancreas graft, lung transplant, intestine transplant and skin graft. In some embodiments the graft is a kidney graft.

In some embodiments, the method has at least 56% sensitivity. In some embodiments, the methods have at least 78% sensitivity. In some embodiments, the methods have a specificity of about 70% to about 100%. In some embodiments, the methods have a specificity of about 80% to about 100%. In some embodiments, the methods have a specificity of about 90% to about 100%. In some embodiments, the methods have a specificity of about 100%.

In another aspect, the invention provides a method for diagnosing or predicting kidney graft status or outcome by determining the presence or absence of a protein recognizing a non-HLA/non ABO antigen, wherein the non-HLA/non ABO antigen is a kidney specific antigen in a sample from a subject who has received a kidney graft. In some embodiments, the protein is an antibody

In some embodiments, the non-HLA/non ABO antigen is a kidney-compartment specific antigen. In some embodiments, the kidney-compartment is selected from the group consisting of renal pelvis, outer cortex, inner cortex, inner medulla, outer medulla, papillary tips and glomeruli. In some embodiments, the kidney-compartment is selected from the group consisting of renal pelvis, outer cortex.

The graft status or outcome may comprises rejection, tolerance, non-rejection based allograft injury, graft function, graft survival, chronic graft injury, or titer pharmacological immunosuppression. In some embodiments, the non-rejection based graft injury is selected from the group of ischemic injury, virus infection, peri-operative ischemia, reperfusion injury, hypertension, physiological stress, injuries due to reactive oxygen species and injuries caused by pharmaceutical agents.

In some embodiments, the sample from a subject who has received a graft is blood, serum, urine, or a stool sample.

In some embodiments, the non-HLA/non ABO antigen is selected from the group consisting of ARHGEF6, PPFIBP2, NIF3L1, ANXA10, STMN3, FAH, SLC6A6, CISD1, CYP4F11, PEX7, PECI, PMM1, IYD, CTNND1, CLIC2, PARVA, CMAH, FOXI1, MFI2, HSPA2, CLDN1, HCFC1R1, MYL4, MPZL2, AFAP1L2, GMPR, MGAT4B, OCLN, MFI2, TMEM61, and PKCζ. In some embodiments, the non-HLA/non ABO antigen is selected from the group consisting of ARHGEF6 and STMN3. In some embodiments, the non-HLA/non ABO antigen is PKCζ.

In another aspect, the invention provides methods for screening and identifying protein recognizing a non-HLA/non ABO antigen that can be useful in the methods described herein, e.g. diagnosing or predicting graft status or outcome. In some embodiments, the protein recognizing a non-HLA/non ABO antigen is an antibody. In some embodiments, the non-HLA/non ABO antigen is a graft specific antigen. In some embodiments, the non-HLA/non ABO antigen is a graft compartment specific antigen.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description. All publications, patents, and patent applications mentioned in this specification are herein incorporated by reference to the same extent as if each individual publication or patent application was specifically and individually indicated to be incorporated by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates enrichment of alloantigenic targets for 7 kidney compartments across 18 kidney transplant patients. Each patient is represented by a similarly colored circle. The location of the circles (displayed on the nephron structure) indicates the specificity of the antigenic response to a particular compartment of the kidney. The larger filled circle indicates the highest antibody response; the smaller filled circle indicates the next highest antibody response. Big empty circles indicate over-enrichment at lower levels. Most patients had over-enrichment with highest level antibodies targeting the renal pelvis, with outer cortex as the next highest compartment. Background image is from the 20th U.S. edition of Gray's Anatomy of the Human Body and is in the public domain (Gray H (1918) PHILADELPHIA: LEA & FEBIGER TWENTIETH EDITION).

FIG. 1B illustrates rank order and antibody signal intensity for post-transplant serological responses across the 7 kidney compartments. The top 5 alloantigenic targets are listed for each compartment.

Left: Rank order (X axis) for post-transplant serological responses across the 7 kidney compartments (Y axis). The highest detection of antibody immune response is ranked for each of the 7 compartments. Each double solid circle indicates that the signal rank was detected as a significant enrichment level across all 18 patient samples. The dashed line with an arrow indicates that the span of ProtoArray targets until antibody detection. Right: The average signal intensity for antibody immune responses for each of the 7 compartments is shown across all 18 patient samples. Each bar represents the average ±standard error of immune response signal intensity. The targets were selected by meeting the criteria a). Antibody immune response signal intensity was positive for at least 70% samples; and b) Coefficient of variation across all 18 samples was less than 1.7. The top 5 targets are listed next to the corresponding kidney compartment. Only 3 targets met these criteria for the inner medulla. The antibodies marked with a star (ARHGEF6 and STMN3) were further selected for validation studies for compartmental localization of the protein in the kidney by immunohistochemistry.

FIG. 2 illustrates enrichment of post-transplant serological responses, specific to kidney compartments, and two control tissues, heart, and pancreas. This graph displays the −log p values of representative post-transplant antibody responses by hypergeometric analysis (patient ID=15) (Y axis) against each of the 7 kidney compartments and heart and pancreas as arbitrary control tissues. These values were plotted against a series of ranked ProtoArray antibody intensities, at every 50 consecutive measurements (X axis). Negative log p=1.3 (a solid horizontal brown line) indicates p=0.05. The top 100 antibodies by intensity are statistically significantly over-enriched with targets expressed in the renal pelvis (purple line rises above horizontal). All other patients are shown in FIGS. 7A-7Q.

FIG. 3 illustrates IHC staining for ARHGEF6 and STMN3 on control kidney tissue. Cytoplasmic staining is observed in the pelvic urothelium with ARHGEF6 and STMN3. Glomerular staining is also observed for ARHGEF6. Left: ARHGEF6 shows positive staining in renal pelvis and glomerulus. Faint staining is seen in proximal tubules, a subset of podocytes and parietal epithelial cells. Right: STMN3 shows positive staining exclusively in the Pelvis: Mild staining, just above background, was seen in proximal tubules, a subset of podocytes and parietal epithelial cells.

FIG. 4 illustrates an integrative genomics flow chart. Work flow (step 1-10) for identifying alloantigenic targets.

FIG. 5 depicts the frequency of compartment-specific allo-antibodies and p value of enrichment across different kidney compartments at multiple ProtoArray thresholds. The data is shown for a single representative patient (ID=15).

FIG. 6 depicts patient demographic information for 36 paired samples (pre- and post-transplant) from 18 kidney transplant recipients (ID). Numerical classification for race and cause of end stage renal disease (ESRD) is shown below. The sample date is shown in months (mos) and the calculated creatinine clearance (CrCl) is based on the Schwartz formula (Schwartz G J et al. (1976) Pediatrics 58, 259-263). Race: 1=Caucasian; 2=Hispanic; 3=Asian; 4=African American; 5=Other. ESRD: 1=Glomerulonephritis; 2=Polycystic Kidney Disease; 3=Dysplasia; 4=Reflux Nephropathy; 5=Obstructive Uropathy; 6=Other.

FIGS. 7A-7Q depict alloantigenic target enrichment by rank antibody levels on 7 kidney compartments for the other 17 patients. Enrichment of post-transplant serological responses, specific to kidney, heart, and pancreas. This graph displays the −log p values of representative post-transplant antibody responses by hypergeometric analysis (the other 17 patients) (Y axis) against each of the 7 kidney compartments and heart and pancreas as arbitrary control tissues. Values were plotted against a series of ranked ProtoArray antibody intensities, at every 50 consecutive measurements (X axis). Negative log p=1.3 (a solid horizontal brown line) indicates p=0.05.

FIG. 8 illustrates allo-antigenic targets by anatomic regions mapped between cDNA microarray and ProtoArray Human Protein Microarrays.

FIG. 9 illustrates patient demographics and association of clinical variables between posttransplant patients who developed acute allograft rejection and posttransplant patients who did not develop acute allograft rejection. HLA, human leukocyte antigen. P-values <0.05 represent a significant difference between the two groups (by independent t-test for continuous variables and by χ²-test for categorical variables. The values are expressed as means, standard deviations, and percentages.

FIG. 10 illustrates an enzyme-linked immunosorbent assay (ELISA) analysis of 15 patients with acute rejection (AR) and 28 stable posttransplant patients. Among the three groups (pre-transplant, posttransplant with AR, and posttransplant stable) there was a non-significant trend toward higher at-event anti-protein kinase C-ζ (anti-PKCζ) levels (P=0.07). The three patients with high anti-PKCζ levels had a mean concentration of 109±34.4 pg/μl. This was more than three times the mean concentration of the remaining 12 patients, that is, 31.1±3.1 pg/μl. This difference was statistically significant (P<0.001). The long horizontal bars represent the mean value for each group and the short horizontal bars represent one standard deviation. None of the patients had high anti-PKCζ(levels pre-transplant (mean value 30.9±5.1 pg/μl). This suggests that the anti-PKCζ response in the three patients is de novo.

FIG. 11 illustrates HLA antibody and biopsy data for patients with high and low anti-PKCζ levels. AR, acute rejection; HLA, human leukocyte antigen; PKCζ, protein kinase C-ζ. Association of HLA antibody and biopsy factors with anti-PKCζ levels. P-values <0.05 represent a significant association between high anti-PKCζ and the respective variable.

FIG. 12 illustrates a Kaplan-Meier analysis of two subtypes of acute rejection (AR) based on serum anti-protein kinase C-ζ (anti-PKCζ) levels. The gray line represents the 12 patients with low serum anti-PKCζ levels and the black line represents the 3 patients with high serum anti-PKC levels. Allograft survival for patients with high anti-PKCζ levels was lower (33%) than that for patients with low anti-PKCζ levels (100%). This was significantly different (P=0.002).

FIGS. 13A-13D illustrate immunohistochemical staining for PKCζ in normal renal tissue and renal parenchyma experiencing acute rejection. Within normal kidney (a and b), cytoplasmic granular staining for PKCζ is observed in a subset of tubules morphologically compatible with distal tubules (b) and the smooth muscle cells of the arteries (a). Patchy endothelial cell staining is observed in a few capillaries. No significant staining is observed in glomeruli except for an occasional infiltrating lymphocyte. In acute rejection (c and d), the tubular staining is less intense, but the infiltrating lymphocytes are PKCζ-positive, both when scattered (d) and when arranged in aggregates (c). Negative controls were run to identify non-specific anti-PKCζ staining. Tissue from non-rejecting allografts had a similar staining pattern to those of normal kidney (data not shown).

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference.

Methods are provided for diagnosing or predicting the graft status or outcome of a subject who has received a graft. The graft status or outcome can comprise rejection, tolerance, non-rejection based graft injury, graft function, graft survival, chronic graft injury, or titer pharmacological immunosuppression.

In some embodiments protein microarrays are used to query de novo or augmented post-graft transplantation antibody responses against non-HLA targets in transplant recipients. The methods described herein allow for the simultaneous interrogation of post-transplant antibody responses to multiple proteins while determining whether the antibody responses are directed against the transplanted graft. Furthermore, the methods described herein allow for the determination of whether post-transplant antibody responses are directed against a specific compartment of the transplanted graft. The advantage of using the methods described herein is that while each graft transplant recipient may have immunogenic antigens in the same compartment of the graft, these specific antigens may not be the same antigens across every patient. Thus, in some embodiments the methods described herein allow for the diagnosis of graft outcomes across all patients who have received a graft by determining whether antibody responses are directed against immunogenic antigen in a specific compartment of the transplanted graft.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In some embodiments, the invention provides methods of determining whether a patient or subject is displaying graft tolerance. The term “graft tolerance” includes when the subject does not reject a graft organ, tissue or cell(s) that has been introduced into/onto the subject. In other words, the subject tolerates or maintains the organ, tissue or cell(s) that has been transplanted to it.

In some embodiments the invention provides methods for diagnosis or prediction of graft rejection. The term “graft rejection” encompasses both acute and chronic transplant rejection. “Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like. Donor-specific antibodies can be a risk factor for acute rejection.

In one embodiment, the rejection is hyperacute rejection. Hyperacute rejection can occur within minutes after the transplantation. Hyperacute rejection can be a complement-mediated response and can result from antibodies against the donor that existed in the host before the transplant.

“Chronic transplant rejection or CR” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

In some embodiments, the invention further includes methods for determining an immunosuppressive regimen for a subject who has received a graft, e.g., an allograft.

Certain embodiments of the invention provide methods of predicting graft survival in a subject comprising a graft. The invention provides methods of diagnosing or predicting whether a graft in a transplant patient or subject will survive or be lost. In certain embodiments, the invention provides methods of diagnosing or predicting the presence of long-term graft survival. By “long-term” graft survival is meant graft survival for at least about 5 years beyond current sampling, despite the occurrence of one or more prior episodes of acute rejection. In certain embodiments, graft survival is determined for patients in which at least one episode of acute rejection has occurred. As such, these embodiments are methods of determining or predicting graft survival following acute rejection. A Kaplan-Meier analysis can be performed to examine graft status or outcomes of patients that express different levels of one or more biomarkers. Graft survival is determined or predicted in certain embodiments in the context of transplant therapy, e.g., immunosuppressive therapy, where immunosuppressive therapies are known in the art.

Immunosuppressive drugs that can be administered to a subject include, for example, glucocorticoids, antibodies, cytostatic agents, and drugs that act on immunophilins. Glucocorticoids can include, for example, prednisolone, prednisone, or methylprednisolone, A cytostatic agent can include, for example, an agent that interferes with nucleic acid synthesis, for example, folic acid, pyrimidine analogs, and purine analogs. A folic acid analog that can be used as an immunosuppressive drug is methotrexate, which can bind dihydrofolate reductase and prevent the synthesis of tetrahydrofolate. Another cytostatic agent is azathioprine, which can be cleaved nonezymatically to form mercaptopurine, which can act as a purine analogue. A cytostatic agent can include, for example, an alkylating agent, including, for example, a platinum compound, cyclophosphamide, and a nitrosourea. Other cytostatic agents include, for example, cytotoxic antibiotics, including dactinomycin, anthracylcines, mitomycin C, bleomycin, and mithramycin. Examples of antibodies that can be immunosuppressive agents include, for example, heterologous polyclonal antibodies, for example, from rabbit or horse. Other antibodies include monoclonal antibodies directed to specific antigens e.g., T-cell receptor directed antibodies (e.g., OKT3, muromonab, which targets CD3), and IL-2 receptor directed antibodies (e.g., targeting CD25). Drugs that can act on immununophilins include, for example, cyclosporin, tacrolimus (Prograf), Sirolimus (rapamycin, Rapamune). Other drugs that can act as immunosuppressive drugs include, for example, mycophenolate (mycophenolic acid), interferons, opioids, TNF binding proteins, Fingolimod, myriocin, and ciclosporin.

An immunosuppressive therapy can be steroid-free or steroid-based. In one embodiment, a steroid-free immunosuppressive therapy comprises administering tacrolimus and mycophenolate mofetil to a subject. In another embodiment, a steroid-based immunosuppressive therapy comprises administering tacrolimus, mycophenolate mofetil, and prednisone to a subject.

In yet other embodiments, methods of determining the class and/or severity of acute rejection (and not just the presence thereof) are provided. Renal allograft biopsies can be evaluated using, for example, Banff classification (see, e.g., Solez K et al. Am J Transplant 2008; 8: 753-760).

In some embodiments, the invention provides methods for diagnosis or prediction of non-rejection based graft injury. Examples of non-rejection based graft injury include, but are not limited to, ischemic injury, virus infection, peri-operative ischemia, reperfusion injury, hypertension, physiological stress, injuries due to reactive oxygen species and injuries caused by pharmaceutical agents.

As in known in the transplantation field, the graft organ, tissue or cell(s) may be allogeneic or xenogeneic, such that the grafts may be allografts or xenografts. A feature of the graft tolerant phenotype detected or identified by the subject methods is that it is a phenotype which occurs without immunosuppressive therapy, i.e., it is present in a host that is not undergoing immunosuppressive therapy such that immunosuppressive agents are not being administered to the host.

In practicing the subject methods, a subject or patient sample, e.g., cells or collections thereof, e.g., tissues, is assayed to diagnose or predict a graft outcome. One or more samples containing one or more cells, can be isolated from body samples, such as, but not limited to, smears, sputum, biopsies, secretions, cerebrospinal fluid, bile, blood, lymph fluid, urine, feces, vomit, cerumen (earwax), gastric juice, breast milk, mucus, saliva, semen, vaginal secretion, a lavage of a tissue or organ (e.g. lung) or tissue which has been removed from organs, such as kidney, breast, lung, intestine, skin, cervix, prostate, pancreas, heart, liver and stomach. For example, a tissue sample can comprise a region of functionally related cells or adjacent cells. Such samples can comprise complex populations of cells, which can be assayed as a population, or separated into sub-populations. Such cellular and acellular samples can be separated by centrifugation, elutriation, density gradient separation, size-based separation, filtration, apheresis, affinity selection (e.g., magnetic affinity cell-sorting, (MACS)), panning, fluorescence-activated cell sorting (FACS), centrifugation with Hypaque, etc. By using antibodies specific for markers identified with particular cell types, a relatively homogeneous population of cells can be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Other devices can separate tumor cells from the bloodstream, see Demirci U, Toner M., Direct etch method for microfluidic channel and nanoheight post-fabrication by picoliter droplets, Applied Physics Letters 2006; 88 (5), 053117; and Irimia D, Geba D, Toner M., Universal microfluidic gradient generator, Analytical Chemistry 2006; 78: 3472-3477. Once a sample is obtained, it can be used directly, frozen, or maintained in appropriate culture medium for short periods of time. Methods to isolate one or more cells for use according to the methods of this invention are performed according to standard techniques and protocols well-established in the art.

In some embodiments, the cells used in the present invention are taken from a patient. Cells used in the present invention can be purified from whole blood by any suitable method.

In another embodiment, a sample from a subject is a cell free sample, for example, a serum or plasma sample. The cell-free sample, e.g., serum or plasma, can comprise antibodies. Methods for generating serum or plasma samples are well known by those skilled in the art.

The term “patient” or “subject” as used herein includes humans as well as other mammals, e.g., cows, horses, dogs, rabbits, mice, rats, and cats. The patient or subject can be a male or female, adult or child. The patient or subject can range in age from, for example, 1-20, 5-20, 5-15, 10-20, or 10-15 years old.

In the methods of the provided invention, a sample can be taken from a subject before the subject receives a transplant (pre-transplant) and/or after the subject receives a transplant (posttransplant). A sample can be taken from a subject with a transplant at least 1 day, at least 1 week, at least 1 month, 2 months, 3 months, 4 months, 5 months, 6 months, 10 months, 15 months, 20 months, 24 months, 3 years, 4 years, 5 years, 10 years, 15 years, 20 years, 30, years, 40 years, 50 years, 60 years, 70 years, 80 years or 90 years after the transplant.

In practicing the methods of the invention, the sample is assayed to determine the presence or absence of a protein recognizing a non-HLA/non ABO antigen. In certain embodiments the presence or absence of a protein recognizing only one non-HLA/non ABO antigen is evaluated. In yet other embodiments, the presence or absence of two or more protein recognizing non-HLA/non ABO antigens, e.g., about 3 or more, about 4 or more, about 5 or more, about 6 or more, about 7 or more, about 8 or more, about 9 or more, about 10 or more, about 15 or more, about 20 or more, about 25 or more, about 30 or more, about 35 or more, about 40 or more, about 45 or more, about 50 or more, about 100 or more, about 200 or more, about 500 or more, about 1,000 or more, about 2,000 or more, about 3,000 or more, about 4,000 or more, about 5,000 or more, about 10,000 or more, about 20,000 or more, or about 30,000 or more etc., is evaluated. In one embodiment, the presence or absence of about 1-30,000, about 1-20,000, about 1-10,000, about 1-6,000, about 1-5,000, about 1-4,000, about 1-3,000, about 1-2,000, about 1-1,000, about 1-500, about 1-200, about 1-100, about 1-50, about 1-10, about 1-5, about 10-20, about 10-25, about 10-50, about 10-100, about 10-200, about 50-200, about 50-100, about 100-200, about 100-500, about 100-1,000, about 100-2,500, about 100-5,000, about 100-10,000, about 100-20,000, or about 100-30,000 proteins recognizing non-HLA/non ABO antigens is evaluated. In one embodiment, the presence or absence of a protein can be determined by comparing a sample taken from a subject before the subject receives a transplant and a sample from the subject after the subject receives the transplant.

The presence or absence of a protein recognizing a non-HLA/non ABO antigen can be determined by any method known in the art. Examples of such methods include, but are not limited to, use of a peptide array(s), use of protein arrays, flow cytometry, a binding assay (e.g., a chromatographic assay, batch binding assay, co-immunoprecipitation, GST-pulldown, etc.) mass spectrometry, (e.g., tandem (MS/MS) mass spectrometry, matrix-assisted laser desorption/ionization (MALDI) mass spectrometry), enzymatic assay (e.g., kinase assay, methylation assay, acetylation assay, polymerization assay), chemical-cross-linking, surface plasmon resonance, Edman degradation, Coosmassie staining (e.g., of a protein in an electrophoresed sample), silver staining (e.g., of a protein in an electrophoresed sample), Bio-Rad Protein Assay, Bradford Assay, far-Western blot, and standard immunoassays (e.g., Western blot, ELISA assays). In certain embodiments, the evaluation is made by protein microarray, as that term is employed in the art. A protein array can include, for example, ProtoArray® Protein Microarray v3 or v5.0 from Invitrogen. Peptide arrays can include the PepStar™ and PepSpot™ peptide arrays from JPT. A protein or peptide array can be a custom-made array. Examples of peptide arrays and protein arrays are described, for example, in U.S. Pat. Nos. 5,744,305 and 6,475,809.

A protein or peptide array used in the methods of the provided invention can comprise protein or peptide sequences from proteins expressed throughout an organism. A protein or peptide array can contain proteins or peptides expressed in one or more organs, e.g., kidney, breast, lung, intestine, skin, cervix, prostate, pancreas, heart, liver, or stomach. In one embodiment, a protein or peptide array contains sequences from proteins expressed in the kidney. A protein or peptide array can contain protein sequences expressed in one or more specific sections, regions, or compartments of an organ, e.g., the kidney. In another embodiment, a protein or peptide array contains one or more proteins expressed in one or more of the outer cortex of the kidney, inner cortex of the kidney, outer medulla of the kidney, inner medulla of the kidney, glomerulus of the kidney, renal pelvis, or papillary tip of the kidney.

Proteins on an array can be full length proteins or fragments of proteins. Proteins on an array can be isolated from an organism, organ, or tissue. In one embodiment, the proteins on the array are isolated from a recombinant source. A protein array can contain at least 10, 100, 250, 500, 1,000, 2500, 5,000, 6,000, 7,000, 8,000, 9,000, or 10,000 different proteins. A peptide array can contain at least 100, 1,000, 10,000, 100,000, or 1,000,000 different peptides.

Proteins or peptides on an array can have posttranslational modifications, including, e.g., phosphorylation, selenoylation, sulfation, arginylation, polysialylation, phosphopantetheinylation, pegylation, palmitoylation, oxidation, nitosylation, acylation, acetylation, alkylation, methylation, amidation, biotinylation, formylation, gamma-carboxylation, glycosylation, glycation, glycylation, hydroxylation, iodination, isoprenylation, lipoylation, prenylation, myristoylation, farnesylation, geranylgeranylation, or ADP-riboyslation.

In one embodiment, the presence or absence of one or more proteins recognizing one or more non-HLA/non ABO antigens is determined by comparing a sample from a subject before an allograft to one or more samples taken from the subject after the allograft. In another embodiment, the presence or absence of one or more proteins recognizing one or more non-HLA/non ABO antigens is determined by comparing samples from subjects with an allograft with one type of graft status or outcome to subjects with an allograft with a different type of graft status or outcome. In another embodiment, the one or more proteins comprise antibodies. In another, the allograft comprises a kidney allograft, heart allograft, liver allograft, pancreas allograft, lung allograft, intestine allograft and skin allograft.

In some embodiments, the expression of e.g., expression profile, for one or more non-HLA/non ABO antigens is evaluated, where the term expression profile is used broadly to include a genomic expression profile, e.g., an expression profile of nucleic acid transcripts, e.g., mRNAs, of the one or more genes of interest, or a proteomic expression profile, e.g., an expression profile of one or more different proteins, where the proteins/polypeptides are expression products of the one or more genes of interest. The expression of one or more non-HLA/non ABO antigens can be determined by any method known in the art. In some embodiments, the expression of one or more non-HLA/non ABO antigens is determined by using microarrays. Alternatively, non-array based methods for detecting the levels of one or more nucleic acids in a sample can be employed, including those based on amplification protocols, e.g., Polymerase Chain Reaction (PCR)-based assays, including quantitative PCR, reverse-transcription PCR(RT-PCR), real-time PCR, Taq-Man real-time PCR, digital PCR, and the like. Other techniques for detecting the levels of one or more nucleic acids can include hybridization methods, for example, Southern blot and Northern blot.

Other methods for detecting the levels of one or more nucleic acids in a sample can include DNA sequencing techniques. Examples of sequencing techniques can include, for example, Sanger sequencing, sequencing by synthesis, sequencing by hybridization, and de novo sequencing. Examples of nucleic acid sequencing techniques, e.g., high throughput nucleic acid sequencing techniques, that can be used in the methods of the provided invention include, e.g., Helicos True Single Molecule Sequencing (tSMS) (Harris T. D. et al. (2008) Science 320:106-109); 454 sequencing (Roche) (Margulies, M et al. 2005, Nature, 437, 376-380), which includes pyrosequencing; SOLiD (Sequencing by Oligonucleotide Ligation and Detection) technology (Applied Biosystems); SOLEXA sequencing (Illumina), comprising bridge amplification of isolated nucleic acids on a surface; single molecule, real-time (SMRT™) technology from Pacific Biosciences; nanopore sequencing (Soni G V and Meller A. (2007) Clin Chem 53: 1996-2001); and use of a chemical-sensitive field effect transistor (chemFET) array (for example, as described in U.S. Patent Application Publication No. 20090026082). In one embodiment, cDNA is generated from RNA by reverse transcription, and the cDNA is sequenced. Nucleic acids can be amplified before being sequenced. In one embodiment, RNA is sequenced.

In another embodiment, nucleic acids (RNA or cDNA) can be enumerated using nCounter™ technology from NanoString Technologies, Inc., in which coded nanoreporters hybridize to specific molecules. Reporter probes, systems and methods for analyzing reporter probes, and methods and computer systems for identifying target specific sequences are described in PCT Publication Nos. WO2007076128, WO2007076129, WO2007076132, WO2007139766, and WO2008124847, and in Geiss G K et al. (2008) Nature Biotechnology 26: 317-325, each of which is herein incorporated by reference in their entireties.

Where the expression profile is a protein expression profile, any convenient protein quantitation protocol can be employed, where the levels of one or more proteins in the assayed sample are determined. Representative methods include, but are not limited to: use of a peptide array(s), use of protein arrays, flow cytometry, a binding assay (e.g., a chromatographic technique, batch binding assay, co-immunoprecipitation, GST-pulldown, etc.), mass spectrometry, (e.g., tandem (MS/MS) mass spectrometry, matrix-assisted laser desorption/ionization (MALDI) mass spectrometry), (e.g., kinase assay, methylation assay, acetylation assay, polymerization assay), chemical-cross-linking, surface plasmon resonance, Edman degradation, Coosmassie staining (e.g., of a protein in an electrophoresed sample), silver staining (e.g., of a protein in an electrophoresed sample), Bio-Rad Protein Assay, Bradford Assay, far-Western blot, and standard immunoassays (e.g., Western blot, ELISA assays). In certain embodiments, the evaluation is made by protein microarray, as that term is employed in the art. A protein array can include, for example, ProtoArray® Protein Microarray v3 or v5.0 from Invitrogen. Peptide arrays can include the PepStar™ and PepSpot™ peptide arrays from JPT. A protein or peptide array can be a custom-made array. Examples of peptide arrays and protein arrays are described, for example, in U.S. Pat. Nos. 5,744,305 and 6,475,809.

In some embodiments, one or more of non-HLA/non ABO antigen are a graft specific antigen. Thus, in some embodiments, the presence or absence of a protein recognizing a graft specific non-HLA/non ABO antigen is determined by any method known in the art including the methods described herein. In some embodiments, the protein recognizing a graft specific non-HLA/non ABO antigen is an antibody. Thus, in some embodiments the invention provides methods for determining the presence or absence of an antibody response against a graft specific non-HLA/non ABO antigen. Examples of grafts include, but are not limited to, kidney graft, heart graft, liver graft, pancreas graft, lung transplant, intestine transplant and skin graft.

The non-HLA/non ABO antigen can be a graft compartment specific antigen. That is the antigen can be specific to a specific compartment of the graft. For example when the graft is a kidney graft, the non-HLA/non ABO antigen can be specific to kidney compartments such as renal pelvis, outer cortex, inner cortex, inner medulla, outer medulla, papillary tips and glomeruli. In some embodiments, non-HLA/non ABO antigen is specific to the renal pelvis or the outer cortex.

In some embodiments the non-HLA/non ABO antigen is a graft compartment specific antigen and the protein recognizing the graft compartment specific antigen is an antibody. Thus, in some embodiments the invention provides methods for determining the presence or absence of an antibody response against a graft compartment specific non-HLA/non ABO antigen.

In some embodiments, the invention provides methods for diagnosing or predicting a kidney graft status or outcome by determining the presence or absence of a protein recognizing a non-HLA/non ABO antigen, wherein the non-HLA/non ABO antigen is a kidney specific antigen in a sample from a subject who has received a kidney graft. In some embodiments, the protein recognizing a non-HLA/non ABO antigen is an antibody. In certain embodiments the presence or absence of a protein recognizing only a kidney specific non-HLA/non ABO antigen is evaluated. In yet other embodiments, the presence or absence of two or more protein recognizing kidney specific non-HLA/non ABO antigens, e.g., about 3 or more, about 4 or more, about 5 or more, about 6 or more, about 7 or more, about 8 or more, about 9 or more, about 10 or more, about 15 or more, about 20 or more, about 25 or more, about 30 or more, about 35 or more, about 40 or more, about 45 or more, about 50 or more, about 100 or more, about 200 or more, about 500 or more, about 1,000 or more, about 2,000 or more, about 3,000 or more, about 4,000 or more, about 5,000 or more, about 10,000 or more, about 20,000 or more, or about 30,000 or more, etc., is evaluated. In one embodiment, the presence or absence of about 1-6,000, about 1-5,000, about 1-4,000, about 1-3,000, about 1-2,000, about 1-1,000, about 1-500, about 1-200, about 1-100, about 1-50, about 1-10, about 1-5, about 10-20, about 10-25, about 10-50, about 10-100, about 10-200, about 50-200, about 50-100, about 100-200, about 100-500, about 100-1,000, about 100-2,500, about 100-5,000, about 100-10,000, about 100-20,000, or about 100-30,000 proteins recognizing kidney specific non-HLA/non ABO antigens is evaluated.

In some embodiments, the protein recognizing a non-HLA/non ABO antigen is an antibody and non-HLA/non ABO antigen is a kidney compartment specific antigen. Thus, in some embodiments the invention provides methods for determining the presence or absence of an antibody response against a kidney compartment specific non-HLA/non ABO antigen.

In some embodiments, the non-HLA/non ABO antigen is selected from the group consisting of ARHGEF6, PPFIBP2, NIF3L1, ANXA10, STMN3, FAH, SLC6A6, CISD1, CYP4F11, PEX7, PECI, PMM1, IYD, CTNND1, CLIC2, PARVA, CMAH, FOXI1, MFI2, HSPA2, CLDN1, HCFC1R1, MYL4, MPZL2, AFAP1L2, GMPR, MGAT4B, OCLN, MFI2, TMEM61, and PKCζ. In some embodiments, the non-HLA/non ABO antigen is selected from the group consisting of ARHGEF6 and STMN3. In one embodiment the non-HLA/non ABO antigen is PKCζ.

In some embodiments, the invention provides methods for diagnosing or predicting graft status or outcome by determining the presence or absence of a plurality of antibodies recognizing non-HLA/non ABO antigens using a protein array in a sample from a subject who has received a graft. In one embodiment, the antibodies recognizing non-HLA/non ABO antigens using a protein array in a sample from a subject who has received a graft are selected from antibodies that recognize antigens selected from the group consisting of ARHGEF6, PPFIBP2, NIF3L1, ANXA10, STMN3, FAH, SLC6A6, CISD1, CYP4F11, PEX7, PECI, PMM1, IYD, CTNND1, CLIC2, PARVA, CMAH, FOXI1, MFI2, HSPA2, CLDN1, HCFC1R1, MYL4, MPZL2, AFAP1L2, GMPR, MGAT4B, OCLN, MFI2, TMEM61, and PKCζ. In one embodiment, the antibody recognizing the non-HLA/non ABO antigen is selected from the group antibodies recognizing antigens consisting of ARHGEF6 and STMN3. In one embodiment, antibody recognizes the non-HLA/non ABO antigen PKCζ.

In some embodiments, the methods described herein for diagnosing or predicting graft status or outcome have at least 56%, 60%, 70%, 80%, 90%, 95% or 100% sensitivity. In some embodiments, the methods described herein have at least 56% sensitivity. In some embodiments, the methods described herein have at least 78% sensitivity. In some embodiments, the methods described herein have a specificity of about 70% to about 100%. In some embodiments, the methods described herein have a specificity of about 80% to about 100%. In some embodiments, the methods described herein have a specificity of about 90% to about 100%. In some embodiments, the methods described herein have a specificity of about 100%.

Also provided herein are methods for screening and identifying protein recognizing a non-HLA/non ABO antigen that can be useful in the methods described herein, e.g. diagnosing or predicting graft status or outcome. In some embodiments, the protein recognizing a non-HLA/non ABO antigen is an antibody. In some embodiments, the non-HLA/non ABO antigen is a graft specific antigen. In some embodiments, the non-HLA/non ABO antigen is a graft compartment specific antigen.

Proteins recognizing graft-compartment specific non-HLA/non ABO antigens can be identified by the methods described in the Examples. After identifying these proteins, one can examine the proteins recognizing these non-HLA/non ABO targets for their correlation with graft status and outcomes such as chronic graft injury, rejection, and tolerance. In some embodiments, the longitudinal change of these proteins recognizing graft-compartment specific non-HLA/non ABO antigens is studied. If clinically significant, these levels can be followed to titer pharmacological immunosuppression, or could be studied as a target for depletion.

Also provided are reagents and kits thereof for practicing one or more of the above-described methods. The subject reagents and kits thereof may vary greatly. Reagents of interest include reagents specifically designed for use in production of the above-described determination of the presence or absence of one or more proteins recognizing a non-HLA/non ABO antigen or the expression profiles of non-HLA/non ABO antigens.

One type of such reagent is an array of probe proteins or peptides in which the non-HLA/non ABO antigens of interest are represented. A variety of different array formats are known in the art, with a wide variety of different probe structures, substrate compositions and attachment technologies. See, for example, U.S. Pat. No. 5,143,854 to Pirrung et al., U.S. Patent Application Publication Nos. 2007/0154946 (filed on Dec. 29, 2005), 2007/0122841 (filed on Nov. 30, 2005), 2007/0122842 (filed on Mar. 30, 2006), and 2008/0108149 (filed on Oct. 23, 2006); Gao et al. “Light directed massively parallel on-chip synthesis of peptide arrays with t-Boc chemistry” Proteomics 2003, 3, 2135-2141; and Ishikawa (WO/2000/003307) “MASKLESS PHOTOLITHOGRAPHY SYSTEM.”

The kits of the subject invention may include the above-described arrays. Such kits may additionally comprise one or more therapeutic agents. The kit may further comprise a software package for data analysis, which may include reference profiles for comparison with the test profile. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such kits may also include instructions to access a database. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1

### Kidney Compartment-Specific Alloimmune Non-HLA Targets can be Identified After Renal Transplantation

### Abstract

We have conducted a novel integrative genomics analysis of serological responses to non-HLA targets after renal transplantation, with the aim of identifying the tissue specificity and types of immunogenic non-HLA antigenic targets after transplantation. Post-transplant antibody responses were measured by paired comparative analysis of pre- and post-transplant serum samples from eighteen pediatric renal transplant recipients, measured against 5,056 unique protein targets on the ProtoArray platform. The specificity of antibody responses were measured against gene expression levels specific to the kidney, as well as two other randomly selected organs (heart and pancreas), by an integrate genomics methodology, employing the mapping of transcription and protoarray platform measures using AILUN. Additionally, the likelihood of post-transplant non-HLA targets being recognized preferentially in any of 7 microdisscected kidney compartments, was also examined by integrate genomics. In addition to HLA targets, non-HLA alloimmune responses, including anti-MICA antibodies, were detected against kidney compartment-specific antigens, with highest post-transplant recognition for renal pelvis and cortex specific antigens. The compartment specificity of selected was confirmed by IHC in normal kidney tissue. In conclusion, this study provides an immunogenic and anatomic roadmap of the most likely non-HLA antigens that can generate serological responses after renal transplantation. Correlation of the most significant non-HLA antibody responses with transplant health and dysfunction are currently underway.

### Methods

### Patients and Samples

Thirty-six paired pre- and post-transplant serum samples from 18 pediatric kidney allograft recipients were included (FIG. 6). All 18 pediatric patients were treated with a steroid-free immunosuppressive protocol (Sarwal M M et al. (2003) Transplantation 76, 1331-1339). Mean age of the patients at transplantation was 11.0±5.5 (range 1-19 years). Twenty-two percent of patients were female, and 67% patients received a kidney from a living donor. The pre-transplant serum samples were collected between August 2001 and April 2006, at 0.6±0.7 (range 0-2.7) months prior to the time of transplantation. The post-transplant serum samples were collected between February 2004 and November 2006, at 24.8±20.8 (range 3-72 months) months after transplantation, as part of the routine follow-up after transplantation. The mean calculated creatinine clearance (Schwartz G J et al. (1976) Pediatrics 58, 259-263) was 99.0±26.8 ml/min/1.73 m² at the time of post-transplant sample collection. Written informed consent was obtained from all subjects and the study was approved by the Institutional Review Board of Stanford University.

### Plasma Profiling Using the Protein Microarray

Serum antibodies were profiled using Invitrogen ProtoArray® Human Protein Microarray v3.0 technology (Invitrogen, Carlsbad, Calif.). This platform contains 5,056 non-redundant human proteins expressed in a baculovirus system, purified from insect cells and printed in duplicate onto a nitrocellulose-coated glass slide. Five mL serum diluted in PBST buffer at 1:150 was applied for 90 minutes onto the ProtoArray, after blocking with blocking buffer for 1 hour. The slides were then washed with 5 ml fresh PBST buffer, 4 times for 10 minutes each, and probed with secondary antibody (goat anti-human Alexa 647, Molecular Probes, Eugene, Oreg.) for 90 minutes. Finally, after a second washing with PBST buffer, the slides were dried and scanned using a fluorescent microarray scanner (GSI Luminoics Perkin-Elmer scanner). All steps were carried out on a rotating platform at 4° C.

### ProtoArray Data Acquisition and Measurement

The slides were scanned at a PMT gain of 60% with a laser power of 90% and a focus point of 0 μm. Fluorescence intensity data were acquired using GenePix Pro 6.0 software (Molecular devices, Sunnyvale, Calif.) with the appropriate “.gal” file downloaded from the ProtoArray central portal on the Invitrogen website (http://www.invitrogen.com/ProtoArray) by submitting the barcode of each ProtoArray slide.

Each protein is spotted twice on each array, to measure the quality of the signal intensity. Pearson correlation coefficients between duplicated spots across all proteins were calculated, and r was over 0.87 for all patients. In addition, standard deviations for duplicated spots for each protein were calculated (Zhu X et al. (2006) Genome Biol 7, R110) (Hudson M E et al. (2007) Proc Natl Acad Sci USA 104, 17494-17499). The standard deviations were decreased by two folds comparing to Immune Response_{Ab} signal (defined below) on the ProtoArray. Given both indications that there was good experiment quality control for duplication, we averaged the values from both spots.

The signal intensity was measured by subtracting the antibody signal detected from the background signal (Signal_{used}=Signal_{Ab}−Signal_{background}) (FIG. 4: step 1). De novo antibody formation after transplantation was identified by using the equation (FIG. 4: step 2)

Immune Response_{Ab}=Signal_{used post-transplant}−Signal_{used pre-transplant}

De novo antibody formation was considered present if Immune Response_{Ab} was positive; negative Immune Response_{Ab} values were eliminated from further analysis.

### Determining Kidney Compartment and Control Organ Specific Gene Expression

Compartmental gene expression measurements from normal kidney tissue (inner and outer cortex, inner and outer medulla, papillary tips, renal pelvis and glomeruli) were previously published by Higgins, et al (Higgins J P et al. (2004) Mol Biol Cell 15, 649-656), using cDNA microarray slides printed at the Stanford Functional Genomics Facility, containing ˜28,000 unique characterized genes or EST's represented by a total of 41,859 unique cDNAs (FIG. 4: step 3). The expression data sets of seven kidney compartments (glomeruli, inner cortex, outer cortex, inner medulla, outer medulla, papillary tip and pelvis) were downloaded from SMD. We first restricted to the published filtered list of 16,293 significant cDNA probes. SAM (Tusher V G et al. (2001) Proc Natl Acad Sci USA 98, 5116-5121) two-unpaired class and multi-class analyses were performed to identify compartmental specific genes on cDNA microarray platform. We first compared the gene expression profiles of each individual compartment with the other compartments all considered together by using a two-class unpaired test, then compared the gene expression profiles of each individual compartment with other different compartments all considered separately using multi-class tests. Both of these were performed using SAM 3.0 (Tusher V G et al. (2001) Proc Natl Acad Sci USA 98, 5116-5121). Genes were selected by the following criteria: (1) FDR<5% by two-class and multi-class, (2) fold change >1 between the target compartment versus all other compartments, and, (3) two-unpaired class analysis was used to identify up-regulated compartment-specific targets (FIG. 4: step 4).

Previously published control non-kidney tissue sample were obtained from GEO (GSE1133) (Su A I et al. (2004) Proc Natl Acad Sci USA 101, 6062-6067). A total of 3,539 genes from the Affymetrix data sets overlapped with the ProtoArray platform. The t-test with Bonferroni-Dunn correction for multiple testing was performed for each of the two control tissues (heart and pancreas) versus all others, then genes were selected at FDR<5%.

AILUN Gene Re-Annotation and Mapping Across cDNA and Protein Array Platforms

The integration of the ProtoArray data with the earlier cDNA microarray gene expression data is complicated by the persistently evolving knowledge of genome and transcriptome annotations. To overcome this inconsistency, we used AILUN (Chen R et al. (2007) Nat Methods 4, 879) to re-annotate from each platforms' probe IDs to the most recent NCBI Entrez Gene ID. Probes on any platform that non-specifically mapped to more than a single NCBI Entrez Gene were eliminated. For comparisons between the kidney compartment cDNA microarray and control tissue Affymetrix microarray data (Higgins J P et al. (2004) Mol Biol Cell 15, 649-656, Su A I et al. (2004) Proc Natl Acad Sci USA 101, 6062-6067) and the ProtoArray data, probes were only kept and further considered if mappings to genes/antigens were present on both technologies (FIG. 4: step 5 and step 9). Across two platforms, 3,835 genes/proteins were identified, which are considered as the population pool in this study.

### Integrated Bioinformatics and Statistical Analysis

The integrated bioinformatics approach, combining ProtoArray data with gene expression microarray results, was restricted to those antigens measured on the ProtoArray matching genes whose expression was also assessed on the cDNA gene expression platform. As the ProtoArray and gene expression microarray data were not normally distributed, Spearman correlation coefficients were first calculated to test for a general association between gene transcriptional levels and the antibody response levels. We found that each compartment's expression levels were statistically significantly different from each patient's antibody profiles (Kolmogorov-Smirnov two-sample test p<0.001, and non-significant Spearman correlation coefficients).

In order to assess the compartment-specific immunogenicity for a patient, we then ranked all proteins by their numerical antibody response. As this is one of the first uses of ProtoArray in human serum antibody measurements, we had no prior threshold response available to use to determine which antigens could be assessed as targeted and which were negative. We thus assessed ProtoArray measurements across a variety of consecutive thresholds. Multiple antibody level thresholds were serially tested, and arbitrarily at each 50 consecutive interval, the set of serum antibodies measured at higher than threshold were tested for over-enrichment of kidney-compartment specific genes using the hypergeometric statistics distribution. At each of multiple consecutive ProtoArray measurement thresholds, we counted how many antigens showed an antibody response above that threshold and of these, how many of these were genes significantly expressed in each renal compartment. The null-hypothesis is that the fraction of renal pelvis specific genes in the antibody response above a threshold is not more than expected. This significance of over-enrichment can be calculated using the hypergeometric test (Tavazoie S et al. (1999) Nat Genet. 22, 281-285; Curtis R K, Oresic M, & Vidal-Puig A (2005) Trends Biotechnol 23, 429-435) (supplemental text).

Using these counts, we then calculated whether there was an over-enrichment of a compartment within a patient's antibody list at that threshold, using the hypergeometric distribution using the following equation and as previously described (Spellman P T & Rubin G M (2002) Journal of biology 1, 5; Fury W et al. (2006) Conf Proc IEEE Eng Med Biol Soc 1, 5531-5534; Tavazoie S et al. (1999) Nat Genet. 22, 281-285Curtis R K, Oresic M, & Vidal-Puig A (2005) Trends Biotechnol 23, 429-435) (FIG. 4: step 6 and FIG. 5). A p<0.05 was considered corresponding to a significant enrichment for that particular anatomic location at that threshold antibody level.

$$Pr ( k = x ) = f ( k ; N , m , n ) = \frac{( \begin{matrix}m \\ k\end{matrix} ) ( \begin{matrix}N - m \\ n - k\end{matrix} )}{( \begin{matrix}N \\ n\end{matrix} )}$$

k=Frequency of observed hits at a certain threshold;
N=Population pool, 3,835 in this study;
m=Threshold at each 50 interval;
n=Expected observation, 161 for glomeruli, 201 for inner cortex, 9 for inner medulla, 336 for outer cortex, 29 for outer medulla, 466 for pelvis, and 167 for papillary tip.

Spearman correlation coefficients were also calculated between post-transplant sample time and the intensity of the antibody responses to selected protein targets to evaluate if antibody responses change with time post-transplantation.

While we acknowledge that these p-values are not controlled for multiple hypotheses testing, we only used the relative ordering of these values for compartment ranking and discovery.

### Immunohistochemistry Staining

Immunohistochemistry staining was performed on paraffin embedded, formalin fixed normal kidney tissue sampled from a radical nephrectomy performed for renal cell carcinoma. The staining procedure was done along with appropriate positive and negative controls performed on normal tissue microarray. Antibodies directed rabbit anti-human (ATLAS antibodies Inc. Protein Tech Group, Inc) were used. Serial sections of 4 μm were obtained, deparaffinized in xylene, and hydrated in a graded series of alcohol. Heat induced antigen retrieval was carried out by microwave pretreatment in citric acid buffer (10 mM, pH 6.0) for 10 minutes. Both antibodies were used at a dilution of 1:50. Endogenous peroxidase was blocked and the DAKO Envision™ system (DAKO Corporation) was used for detection. The staining was optimized using appropriate positive and negative controls.

### Supplemental Methods

### Plasma Profiling Using the Protein Microarray

Five mL serum diluted in PBST buffer at 1:150 was applied for 90 minutes onto the ProtoArray, after blocking with blocking buffer for 1 hour. The slides were then washed with 5 ml fresh PBST buffer, 4 times for 10 minutes each, and probed with secondary antibody (goat anti-human Alexa 647, Molecular Probes, Eugene, Oreg.) for 90 minutes. Finally, after a second washing with PBST buffer, the slides were dried and scanned using a fluorescent microarray scanner (GSI Luminoics Perkin-Elmer scanner). All steps were carried out on a rotating platform at 4° C.

### ProtoArray Data Acquisition and Measurement

The slides were scanned at a PMT gain of 60% with a laser power of 90% and a focus point of 0 μm. Fluorescence intensity data were acquired using GenePix Pro 6.0 software (Molecular devices, Sunnyvale, Calif.) with the appropriate “.gal” file downloaded from the ProtoArray central portal on the Invitrogen website (http://www.invitrogen.com/ProtoArray) by submitting the barcode of each ProtoArray slide. Each protein is spotted twice on each array, to measure the quality of the signal intensity. The standard deviations were decreased by two folds comparing to Immune Response Ab signal on the ProtoArray. Given both indications that there was good experiment quality control for duplication, we averaged the values from both spots. De novo antibody formation was considered present if Immune Response Ab was positive; negative Immune Response Ab values were eliminated from further analysis.

### Integrated Bioinformatics and Statistical

The integrated bioinformatics approach, combining ProtoArray data with gene expression microarray results, was restricted to those antigens measured on the ProtoArray matching genes whose expression was also assessed on the cDNA gene expression platform. As the ProtoArray and gene expression microarray data were not normally distributed, Spearman correlation coefficients were first calculated to test for a general association between gene transcriptional levels and the antibody response levels.

In order to assess the compartment-specific immunogenicity for a patient, we then ranked all proteins by their numerical antibody response. As this is one of the first uses of ProtoArray in human serum antibody measurements, we had no prior threshold response available to use to determine which antigens could be assessed as targeted and which were negative. We thus assessed ProtoArray measurements across a variety of consecutive thresholds. Multiple antibody level thresholds were serially tested, and arbitrarily at each 50 consecutive interval, the set of serum antibodies measured at higher than threshold were tested for over-enrichment of kidney-compartment specific genes using the hypergeometric test. At each of multiple consecutive ProtoArray measurement thresholds, we counted how many antigens showed an antibody response above that threshold and of these, how many of these were genes significantly expressed in each renal compartment. The null-hypothesis is that the fraction of renal pelvis specific genes in the antibody response above a threshold is not more than expected. This significance of over-enrichment can be calculated using the hypergeometric test.

We calculated whether there was an over-enrichment of a compartment within a patient's antibody list at that threshold, using the hypergeometric distribution using the following equation and as previously described (FIG. 4: step 6 and FIG. 5). For example, 466 genes out of 3,835 mapped targets are expressed in the renal pelvis. In patient ID 15, we see 17 antigens showing an antibody response above the top 100 threshold. The null-hypothesis is that the fraction of renal pelvis specific genes in the antibody response above a threshold is not more than expected. Instead, we see 17 antigens from the renal pelvis, suggesting that genes expressed in the renal pelvis are over-enriched in the antibody response. Spearman correlation coefficients were also calculated between post-transplant sample time and the intensity of the antibody responses to selected protein targets to evaluate if antibody responses change with time post-transplantation.

### Results

Identification of De Novo Non-HLA Non-ABO Antibody Formation after Renal Transplantation

The formation of de novo antibodies after renal transplantation was assessed by comparing 18 post-transplant serum samples with 18 paired pre-transplant serum samples. Of the 5,056 proteins present on the ProtoArray, an average of 61% (range across patients 21%-96%) had an increased signal after transplantation. A complete list of all 5,056 antigens and raw data can be downloaded from the NCBI Gene Expression Omnibus (GEO) http://www.ncbi.nlm.nih.gov/geo (Barrett T, et al. (2007) Nucleic Acids Res 35, D760-765).

Mapping Genes that are Specifically Expressed in Different Compartments of Normal Kidney

We obtained from the Stanford Microarray Database (SMD) (http://med.stanford.edu/jhiggins/Normal_Kidney/download.shtml) a previously reported cDNA microarray gene expression data set, in which 34 samples were obtained from 7 different renal compartments of normal kidneys: glomerular (n=4), inner cortex (n=5), outer cortex (n=5), inner medulla (n=5), outer medulla (n=5), pelvis (n=5) and papillary tip samples (Higgins J P et al. (2004) Mol Biol Cell 15, 649-656). Raw data was downloaded and genes significant for each compartment selected by SAM (Tusher V G et al. (2001) Proc Natl Acad Sci USA 98, 5116-5121), using an FDR<=5%. Probes, specific to each kidney compartment, were retained in our analysis based on the published filtered list of 16,293 cDNA probes from approximately 42,000 total.

Cross mapping kidney compartment specific gene probes to protein targets on the Protoarray to select potential kidney compartment specific proteins, Cross mapping of gene IDs on the gene expression microarray and the ProtoArray platforms was conducted using AILUN software (Chen R et al. (2007) Nat Methods 4, 879). The number of compartment specific genes that were also measured on the ProtoArray platform, are shown in FIG. 8.

Identification of HLA and MICA Antibodies after Kidney Transplantation

Fifty percent of patients (9/18) had showed positive de novo donor specific antibody responses clinically detected by flow cytometry performed at the Stanford histocompatibility lab, detected at a mean time of 24 months post-transplantation. We compared the ability to detect anti-HLA antibodies by ProtoArray measurements, with clinical measurement of anti-HLA antibodies by flow cytometry. On the ProtoArray, there are only four proteins are annotated as major histocompatibility antigens, specific for class I (HLA-B) and class II (HLA-DPA1, HLA-DMA, and HLA-DRA). Despite the paucity of available HLA antigens to interrogate on this platform, five of these 9 patients showed de novo anti-HLA antibody generation against either/or HLA-class I and HLA-class II by ProtoArray, with no false-positives. This yields a calculated performance for detecting anti-HLA antibodies by ProtoArray at 56% sensitivity, 100% specificity, 100% positive predictive value, and 70% negative predictive value. Of note, based on the cross mapping of the protoarray platform by AILUN, there was no specific renal compartment noted to express HLA antigens selectively. Anti-MICA antibodies (MICA=MHC class I polypeptide-related sequence A), previously described to increase in the post-transplant period in patients after transplantation, and often associated with adverse graft outcomes (Terasaki P I et al. (2007) Am J Transplant 7, 408-415; Zou Y et al. (2007) N Engl J Med 357, 1293-1300), was at a higher level after transplantation in 72% of patients in this study. There was no correlation between the mean intensity of post-transplant antibody responses and HLA match or mismatch grades.

Integrative cDNA-Protein Microarray Analysis for Compartment-Specific Immunogenicity

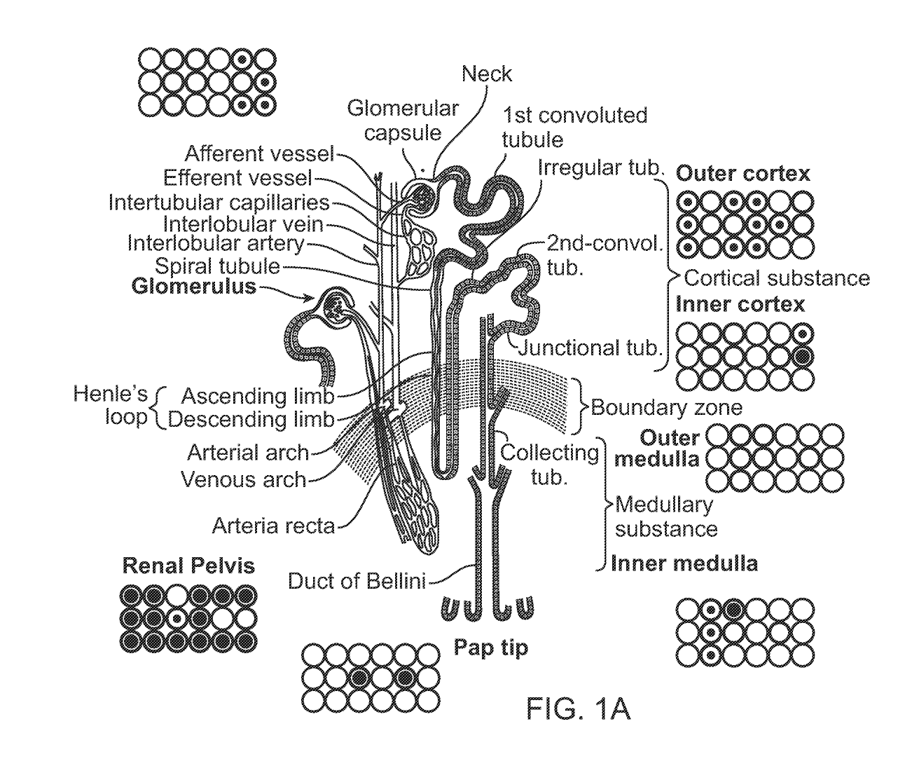

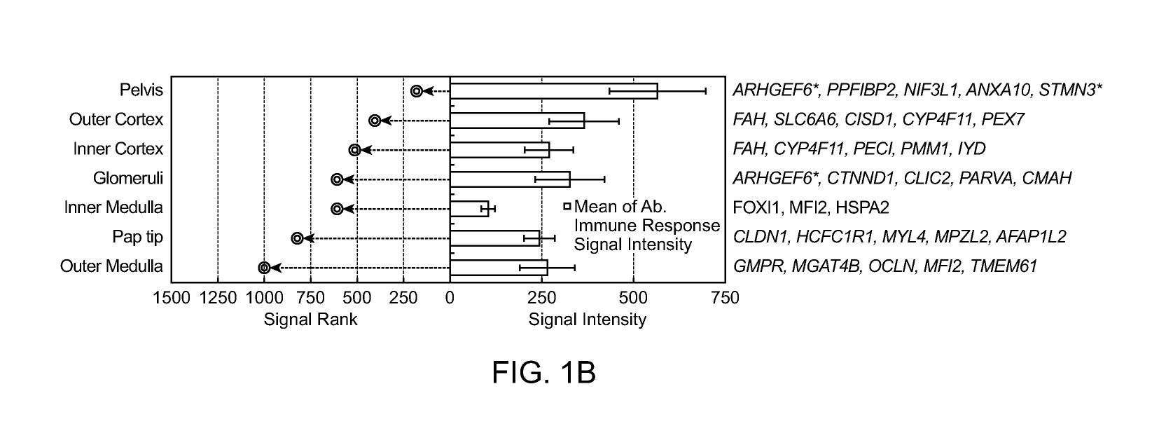

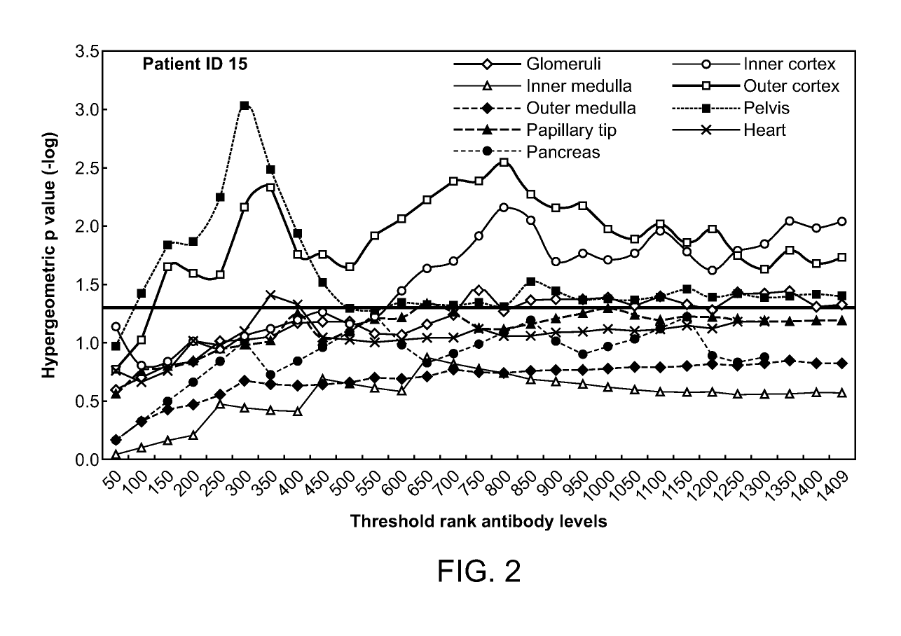

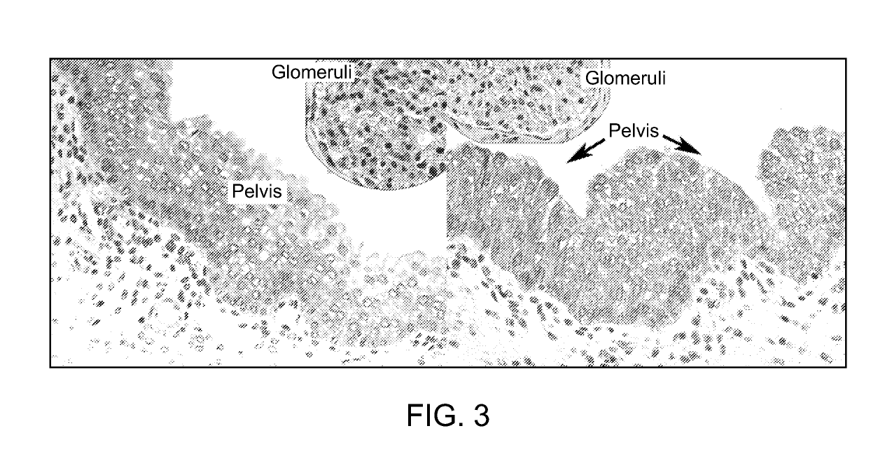

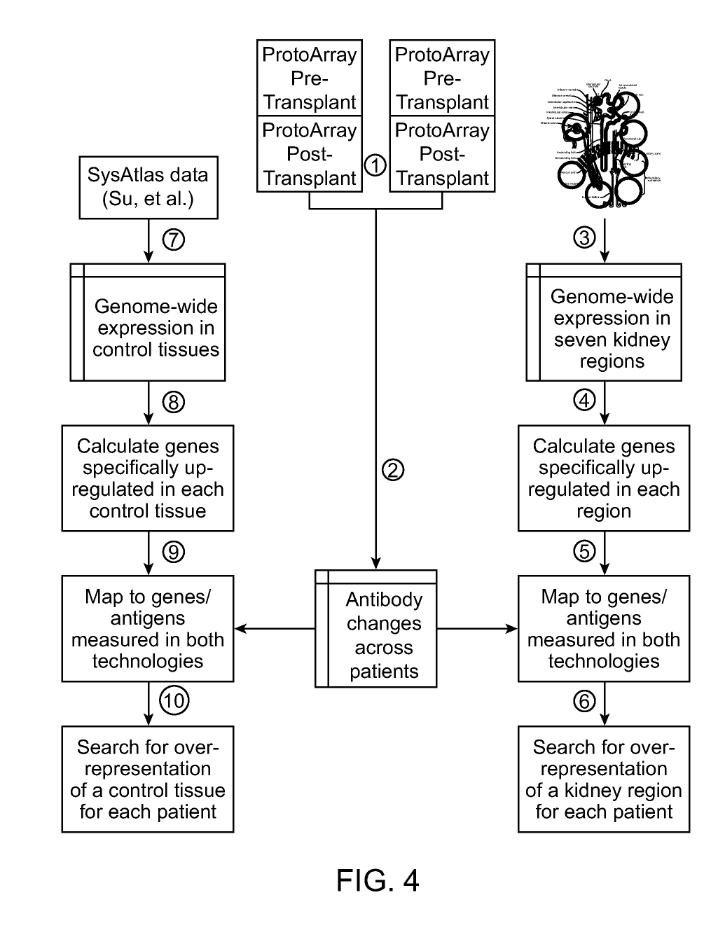

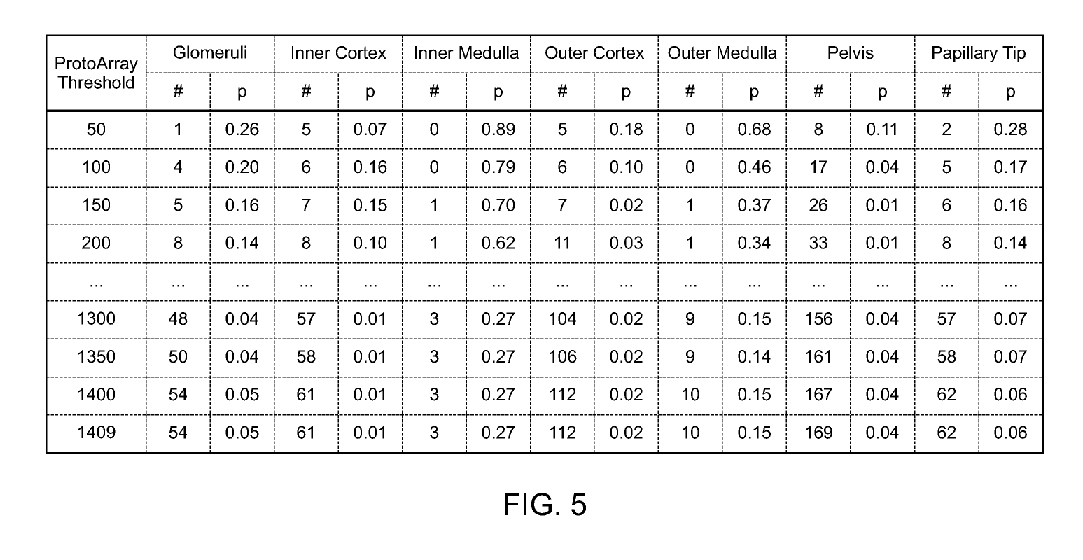

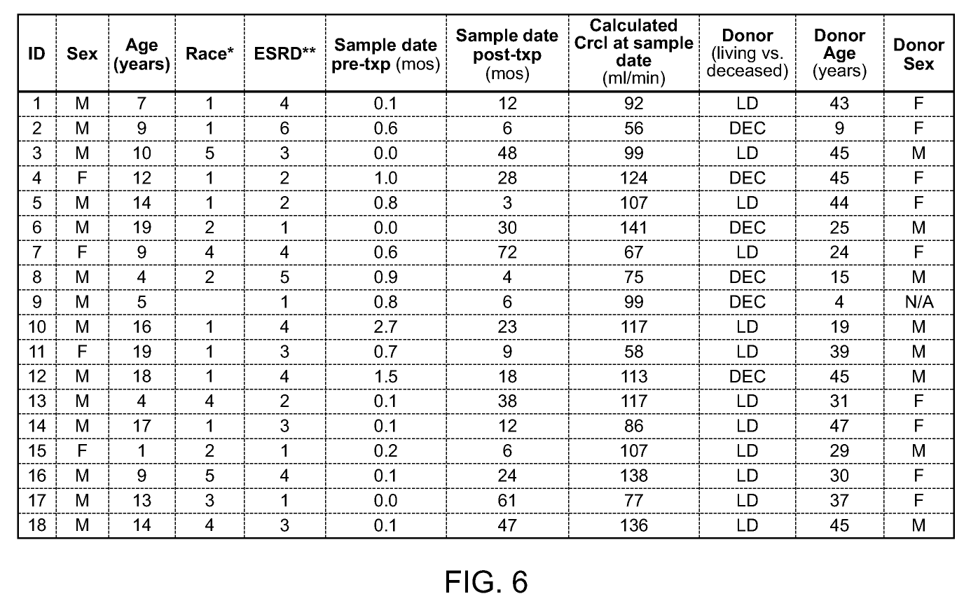

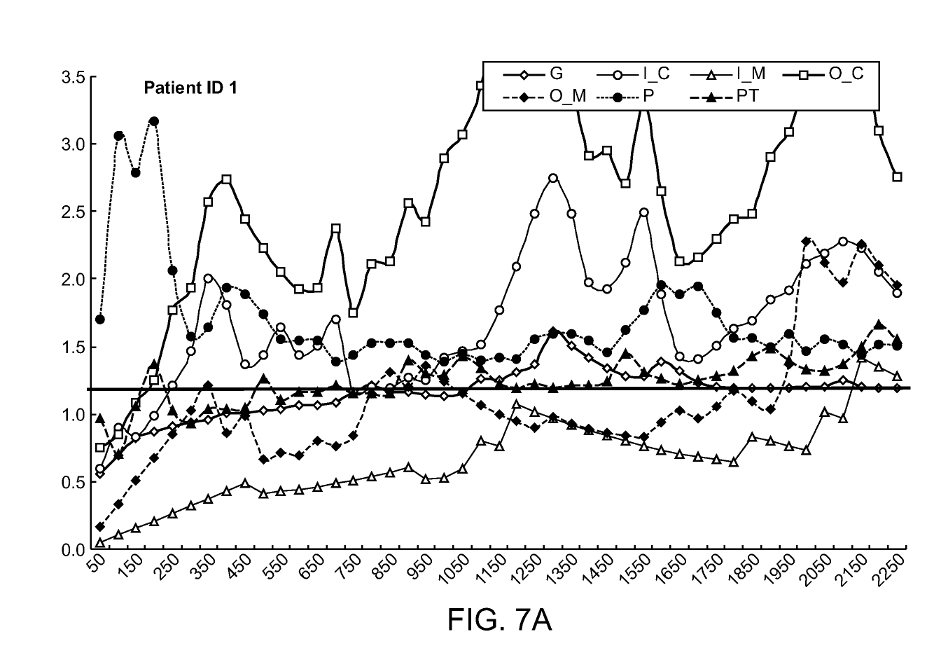

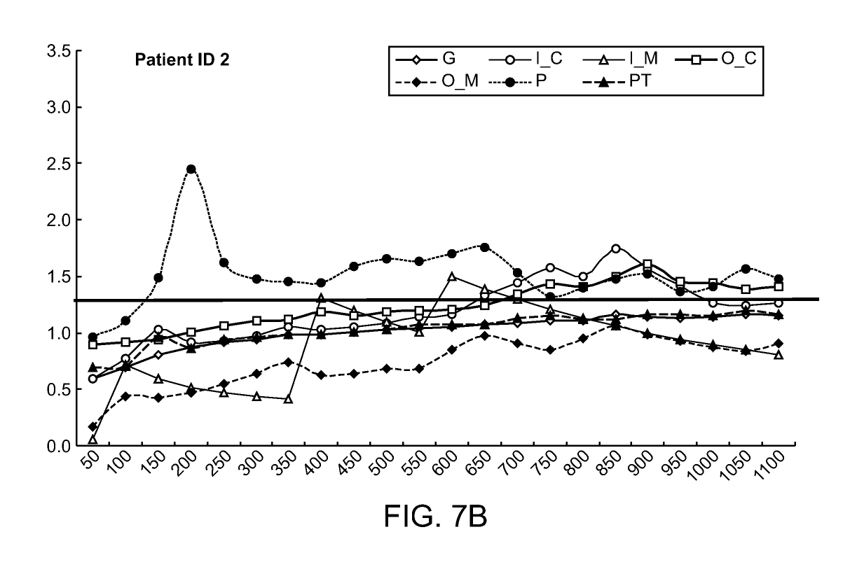

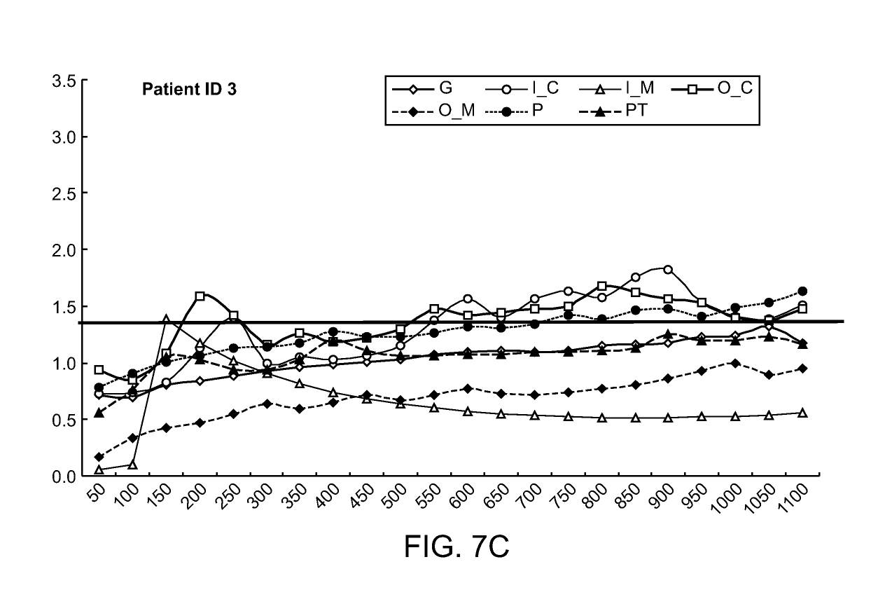

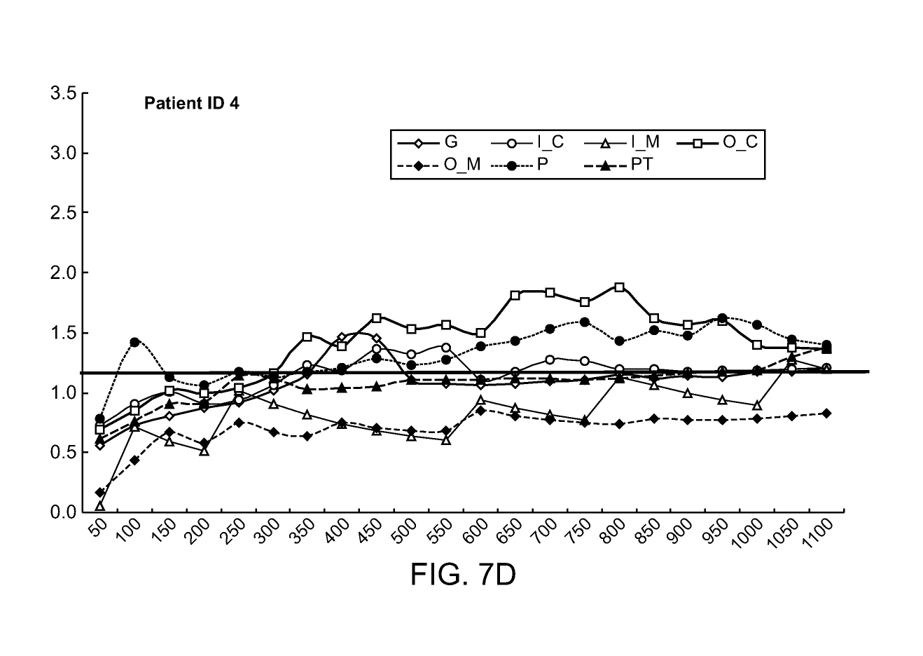

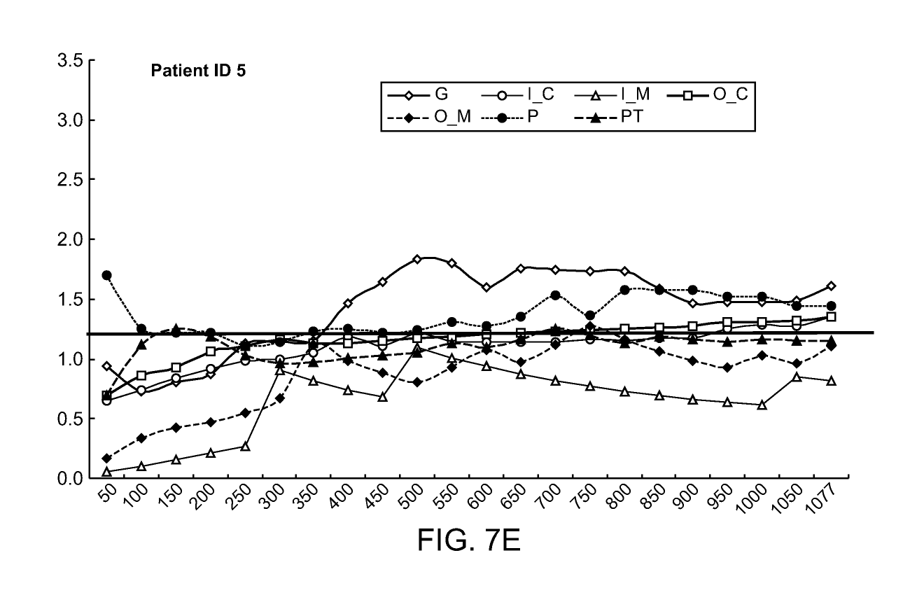

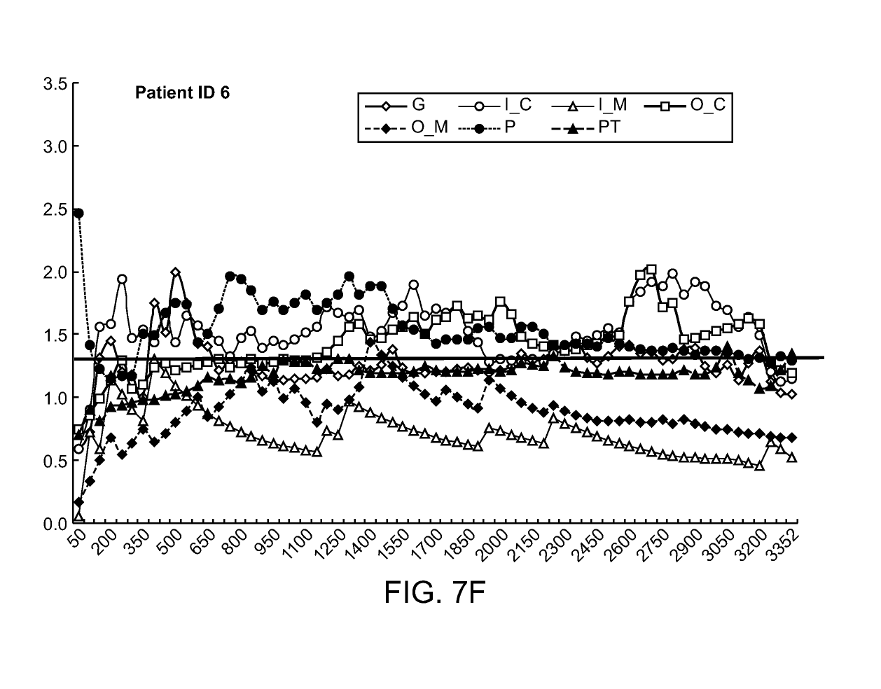

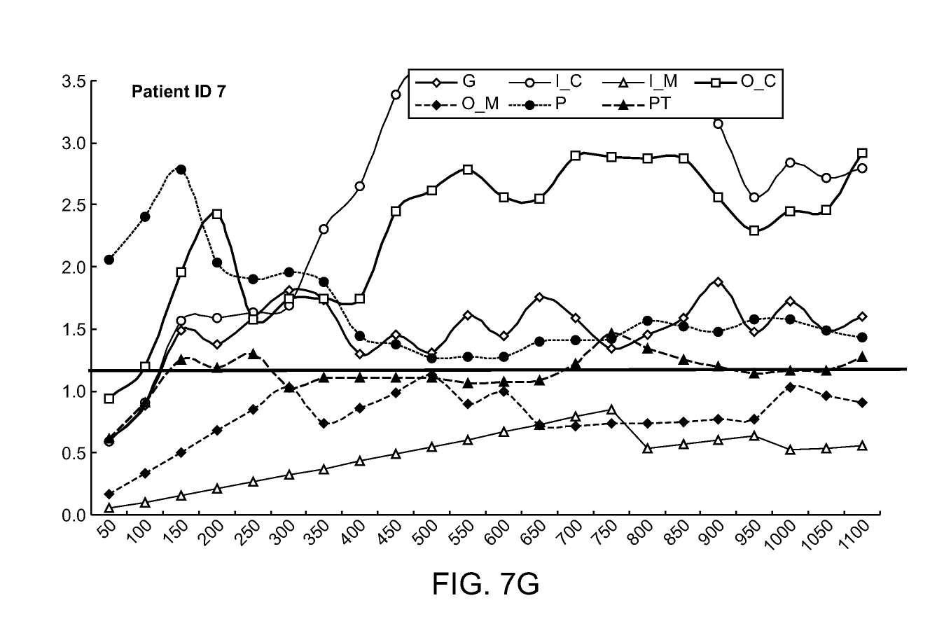

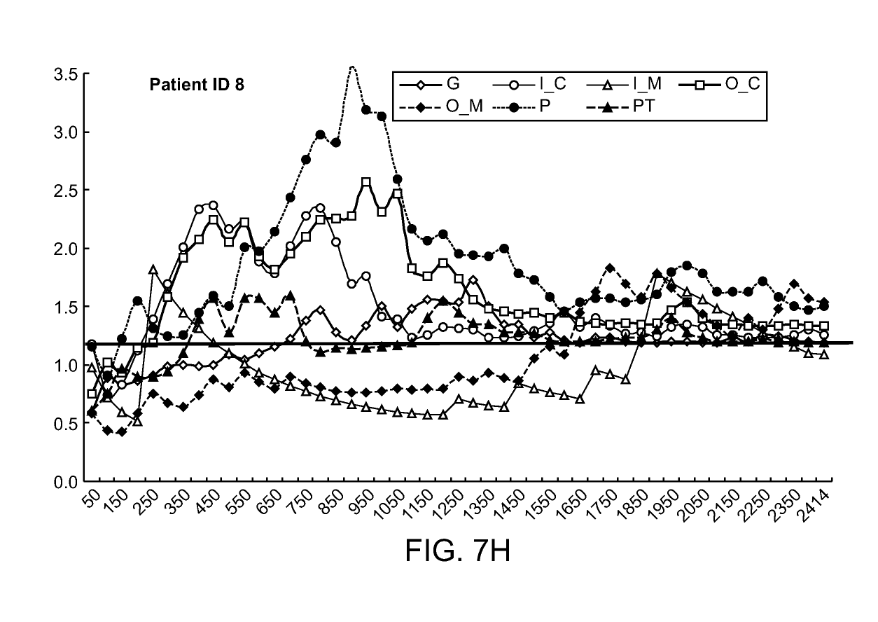

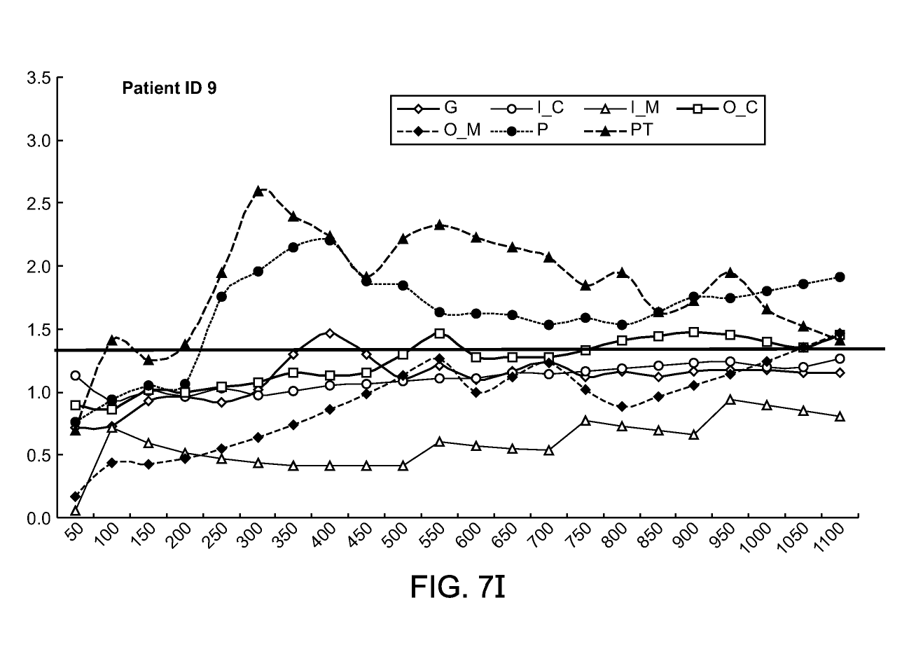

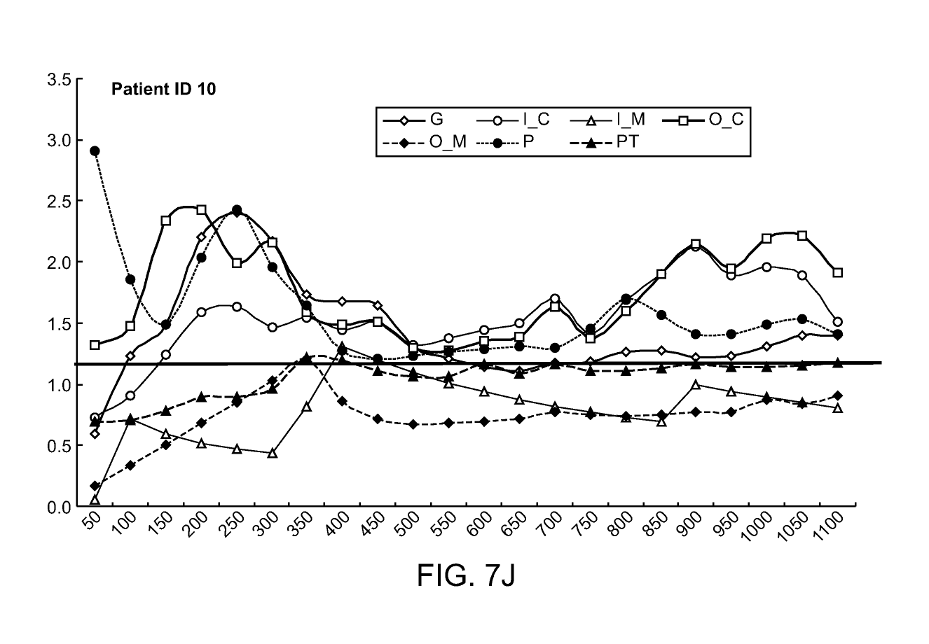

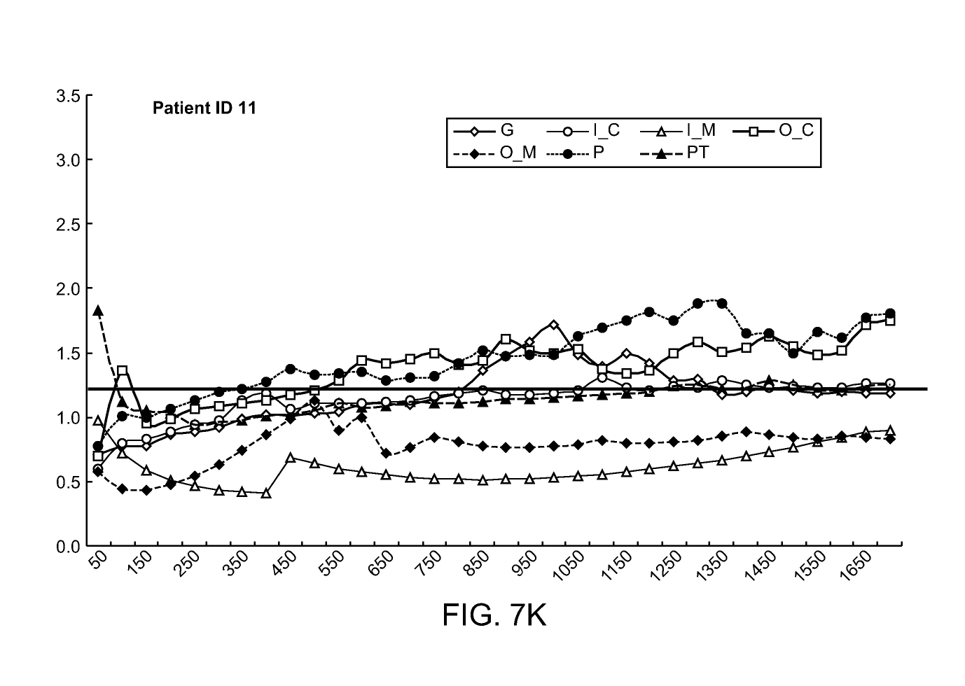

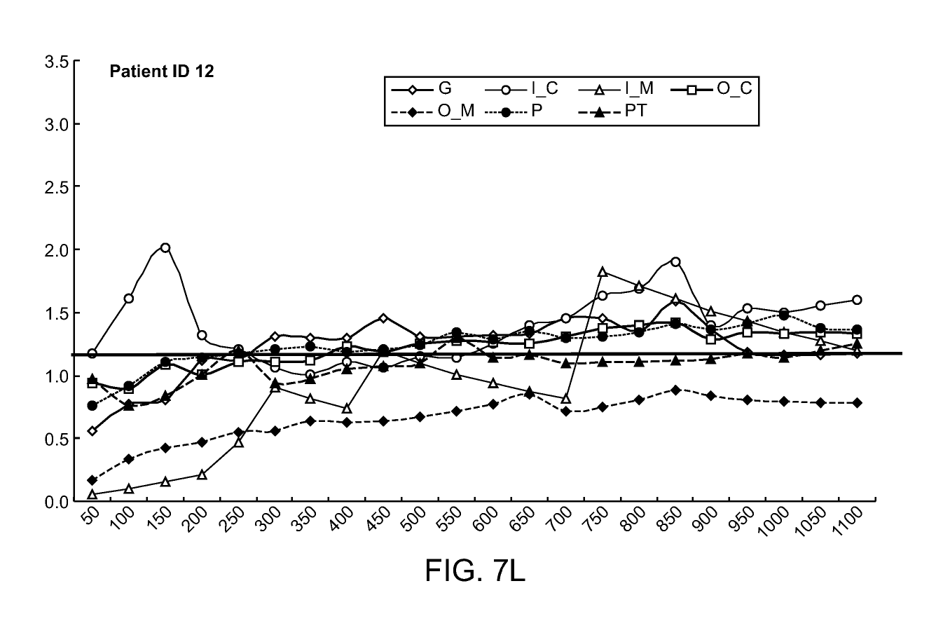

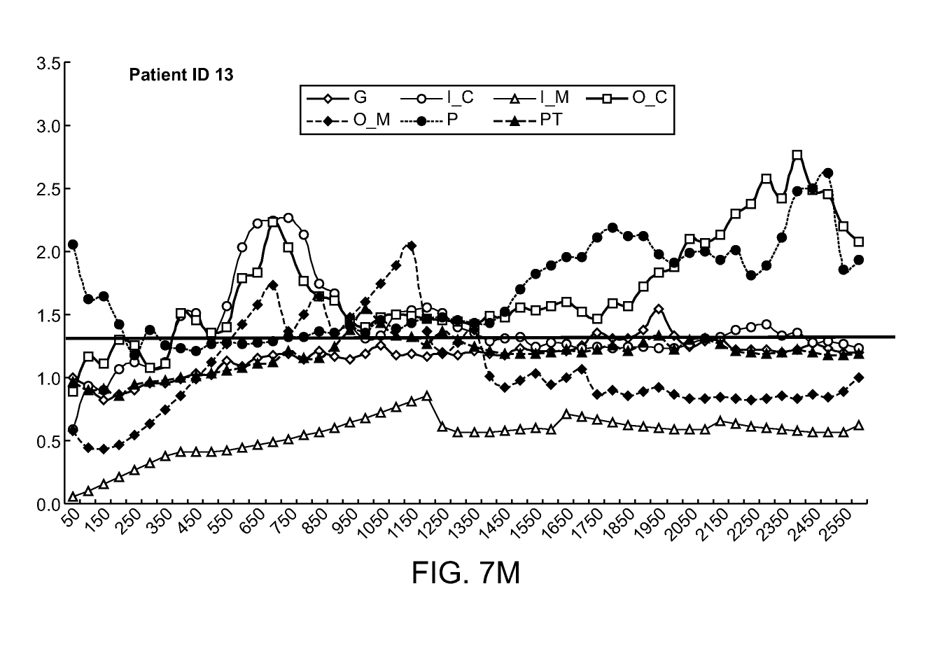

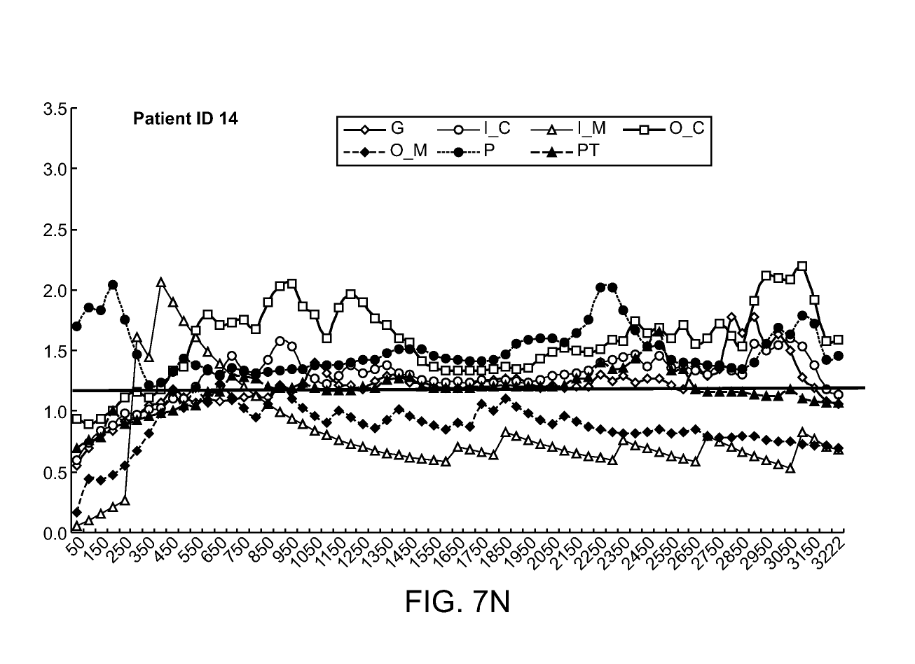

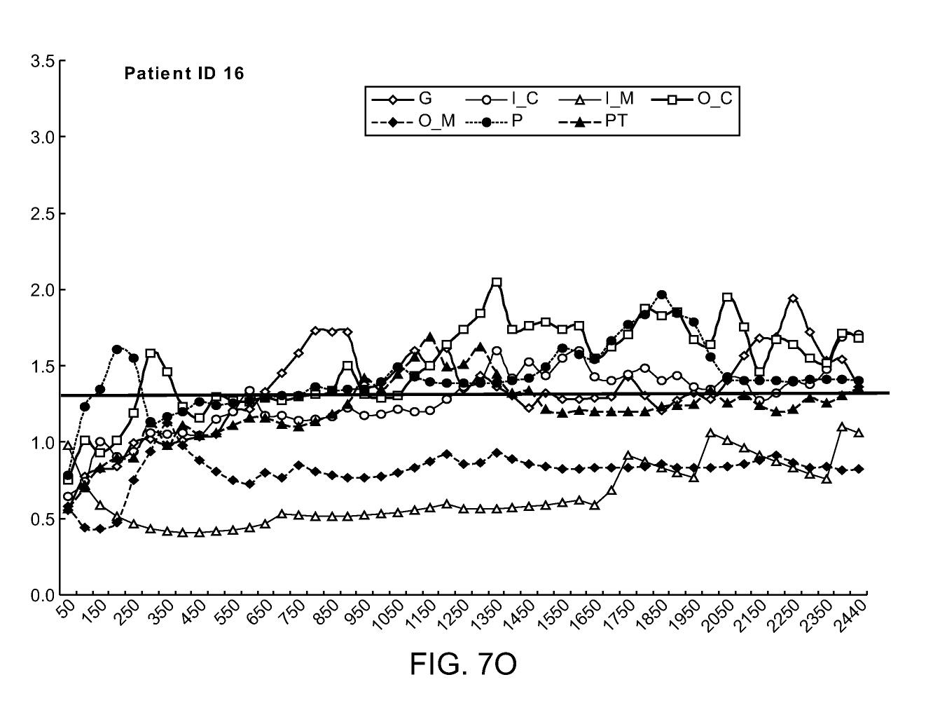

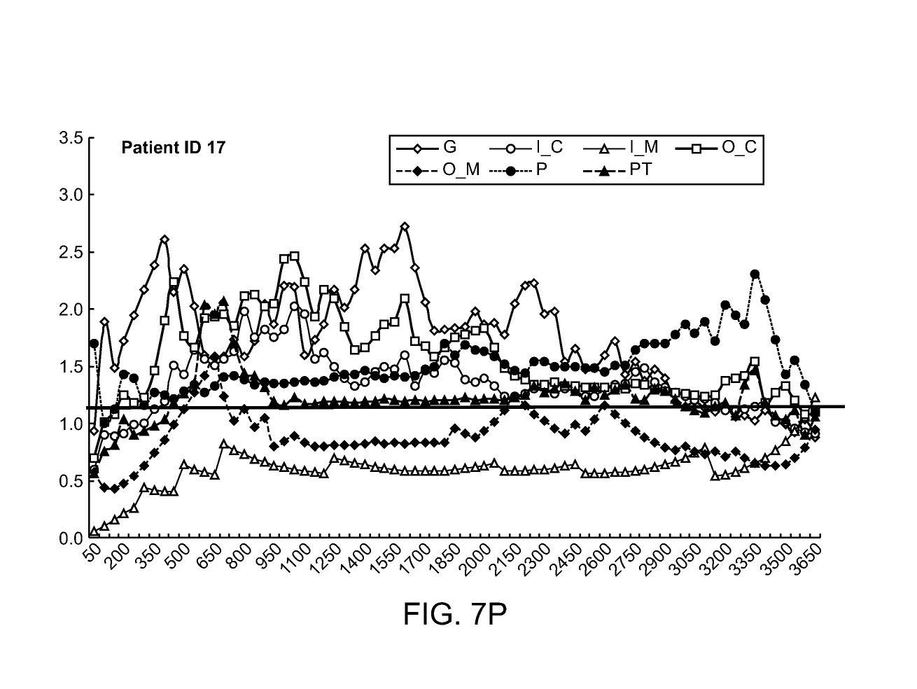

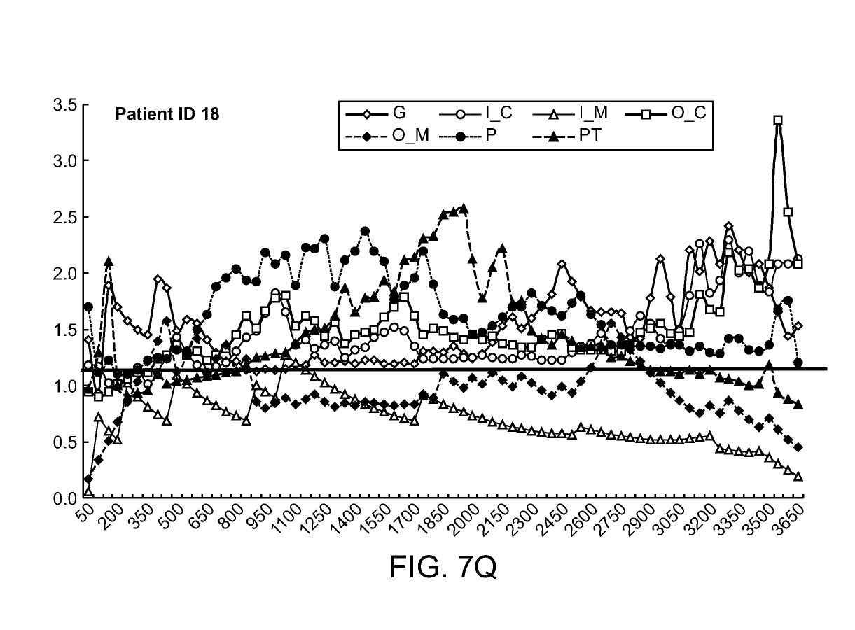

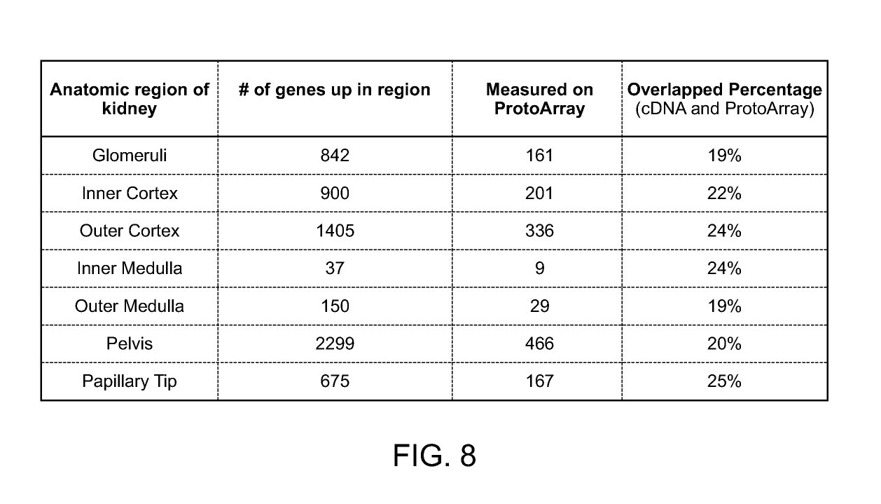

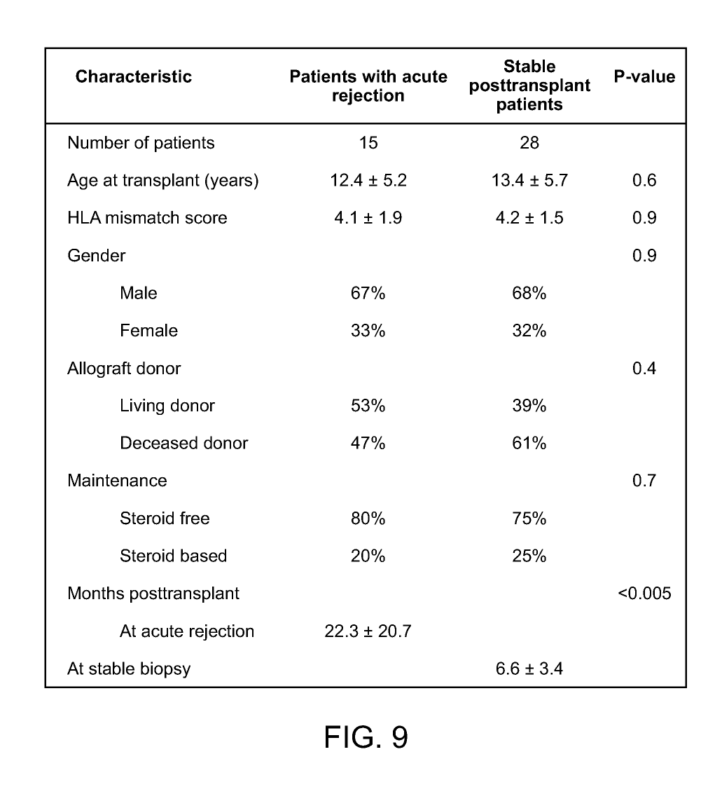

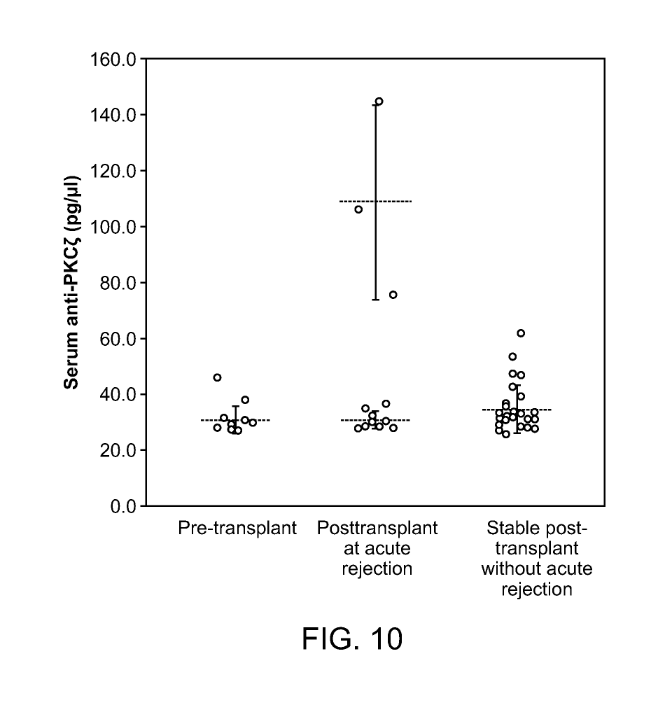

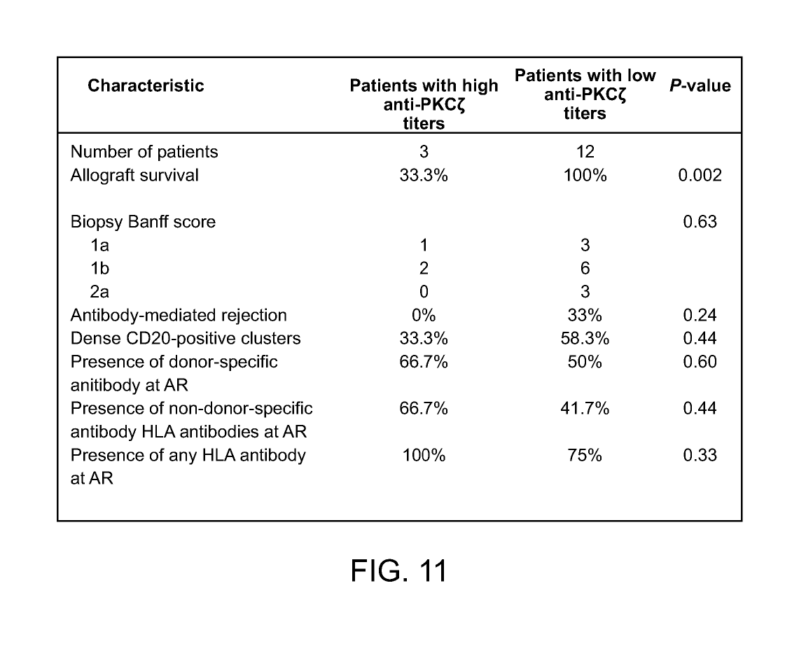

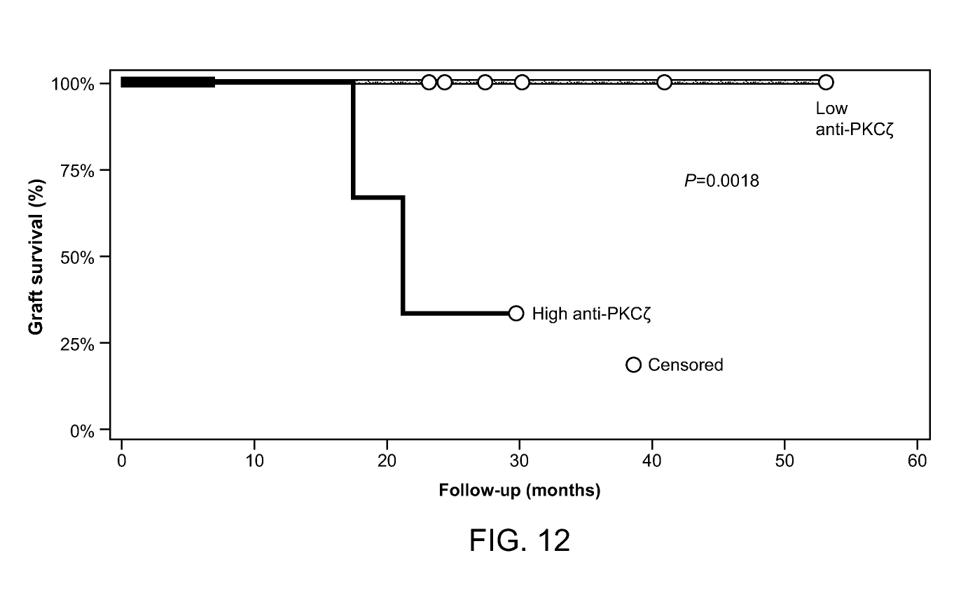

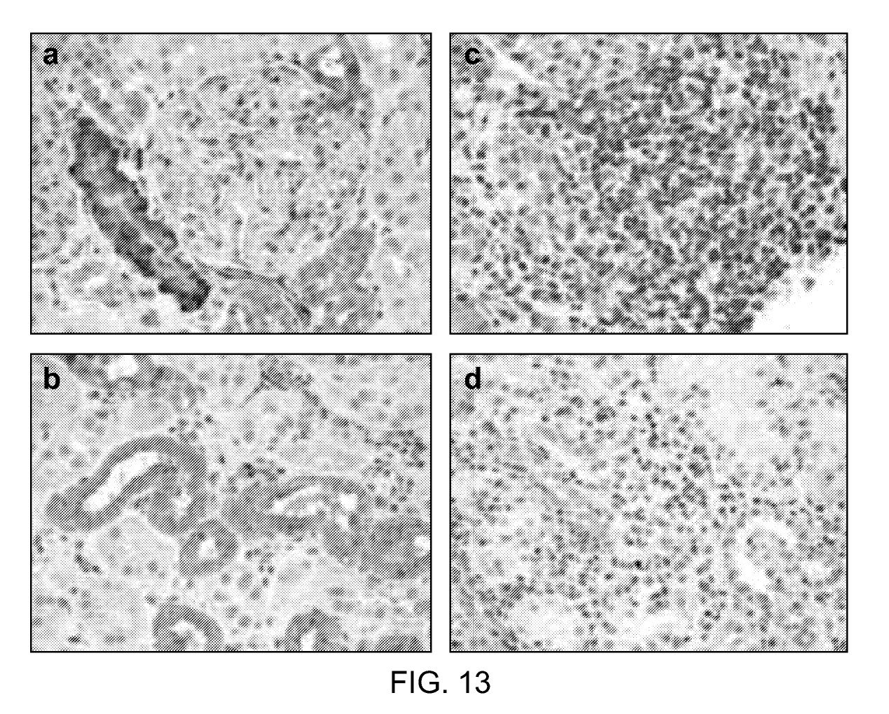

A list of proteins against which each patient developed significant antibody responses was generated for each patient, across a variety of possible threshold levels of immune response signal intensities. Each list was then tested to see whether these antigens were significantly over-enriched by their corresponding genes, expressed in any particular individual renal compartment from the microarray dataset (Higgins J P et al. (2004) Mol Biol Cell 15, 649-656), by utilizing the hypergeometric distribution (FIG. 5). If there was no renal compartment-specific post-transplant antibody targeting, then over-enrichment of compartment-specific antigens would not be expected as the significance threshold of ProtoArray signals were reduced. Contrary to this expectation, we saw significant over-enrichment of compartment-specific antigens in every renal transplant patient. Surprisingly, for 14 out of the total 18 patients (78%), we found that the renal pelvis was the anatomic location showing the most significant enrichment for post-transplant antibody immune responses (FIG. 1A). Based on this approach we can rank each of the seven kidney compartments with regards to their immunogenic potential to mount specific antibody responses to kidney specific targets after transplantation. This ranking can be based on the level of a specific antibody against that compartment, as well as the mean of all antibody levels targeting that compartment. Regardless of the rank method, the highest antibody levels were consistently noted against the renal pelvis (average rank order of highest antibody=167), followed by the outer renal cortex (average rank order of highest antibody=397). When compartment-specific antibody responses were examined overall, the renal pelvis again had the highest average antibody level (564), followed by the outer renal cortex (364) (FIGS. 1A and 1B). While other compartments of the kidney were targeted by antibodies at lower levels, it is noteworthy that antibodies to outer medulla were not noted to be significant in any patient (FIGS. 1A, 2, and 7).

Spearman correlation coefficient analysis between the intensity of post-transplant antibody responses to 5,056 antigenic targets on the ProtoArray and time post-transplantation, shows that though overall many of the antibody responses are associated with post-transplant sample time (r=0.69, p=0.0016), this is independent of the discovery of antibodies generated after transplantation to kidney compartment specific protein targets.

### Specificity of Antibody Responses for Renal Antigens

In order to assess whether the antibodies detected after renal transplantation were indeed specific against targets only expressed in kidney tissue, we used tissue-specific data sets from other organs for comparison. Tissue-specific gene expression data were used from a published study by Su, et al., on 79 human and 61 mouse tissues, hybridized on Affymetrix GeneChip Human Genome U133 Arrays (Su A I et al. (2004) Proc Natl Acad Sci USA 101, 6062-6067). From this data set, we arbitrarily selected organ-specific gene expression profiles for heart and pancreas, and found 122 genes expressed significantly in heart tissue, and 26 in pancreatic tissue. Using a similar approach as previously, there was no significant enrichment by hypergeometric analysis of serological responses against heart or pancreatic tissue-specific antigens in the sera of any of the 18 kidney transplant recipients. Based on these results it appears that kidney compartment-specific non-HLA antigenic targets are specifically recognized and can mount significant antibody responses after kidney transplantation (FIG. 2).

### Immunohistochemistry (IHC) to Confirm Compartment Specific Localization of Antigens in the Kidney

To confirm that compartment-specific serological responses are mounted against specific kidney compartment antigens, we sought to demonstrate if indeed the antigenic localization, predicted by the integrated genomics approach used, could be replicated by immunohistochemical localization of the same antigen in predicted renal compartment. Two antibodies were selected for IHC. One antibody, anti-ARHGEF6, had high post-transplant signals in 100% (18/18) patients and was predicted to be specifically expressed in two compartments of the kidney: the renal pelvis and the glomerulus. IHC confirmed accurate localization of this antigen with positive cytoplasmic staining in the pelvic urothelium, and the glomerulus (FIG. 3: left). The second antibody, anti-STMN3, had increased post-transplant signal intensity in 83% (15/18) patients with a prediction for strong expression in the renal pelvis. IHC again confirmed the predicted localization of this antigen, with positive cytoplasmic staining localized solely to the pelvic urothelium (FIG. 3: right). Thus, it appears that the integrated genomics approach to predict specific tissue localization of genes and proteins in human tissues is accurate and can be validated.

### Discussion

This is the first study to explore the use of high-density protein microarrays to study non-HLA serological responses after kidney transplantation. We demonstrate that ProtoArray measurements reveal increased serological responses that can be recognized in all patients after renal transplantation, across 61% of the targets interrogated on the ProtoArray. To ascertain that these serological responses are specific to the transplanted organ, analysis for over-enrichment was performed using a novel method of integration of these serum antibody measurements with previously published compartment-specific normal kidney gene expression measurements. These studies demonstrated that the post-transplant serological responses detected were in fact selectively recognizing kidney antigens; furthermore these antibody responses were recognizing relevant kidney-compartment specific antigens. We discovered that these post-transplant serological responses were specific to the kidney, and were not noted randomly to other organs such as the heart and pancreas; thus suggesting that these serological responses may in fact be specific to the kidney transplant. The seven different renal compartments studied were found to vary in immunologic potential after transplantation, with the renal pelvis generating the highest levels of compartment-specific antibody responses. Immunohistochemistry based localization for two selected antigens confirmed the predicted tissue localization of the antigens, derived from the integrated genomics approach in this study.

Integrative genomics has been defined as the study of complex interactions between genes, organisms and environment of biological data. Methods in integrative genomics have been previously used to find genes associated with both rare diseases, such as Leigh Syndrome French-Canadian type (Mootha V K et al. (2003) Proc Natl Acad Sci USA 100, 605-610), and common polygenic disorders, such as obesity (English S B & Butte A J (2007) Bioinformatics 23, 2910-2917), given genetic linkage data, proteins identified through mass spectrometry, and gene expression measurements. Here, we are using integrative genomics in a novel manner, using publicly-available histopathological gene expression measurements as a kind of lens, to focus the set of antibody level changes into a specific set relevant to kidney transplantation. The advantage of using an integrative genomics method is that while each patient may have immunogenic antigens in the same compartment of the kidney, these specific antigens may not be the same antigens across every patient. Only by considering the measurements anatomically does one find a consistent pattern across all patients, seen in FIG. 1A.

We find that all 18 patients demonstrate an increase in antibodies against Rac/Cdc42 guanine nucleotide exchange factor 6 (ARHGEF6, also known as PIXα), a protein we show is expressed in the renal pelvis and glomerulus. ARHGEF6 has been previously independently shown to be expressed at a moderate level in human kidney (Kutsche K & Gal A (2001) Cytogenetics and cell genetics 95, 196-201). ARHGEF6 is activated by phosphatidylinositol 3-kinase (Yoshii S et al. (1999) Oncogene 18, 5680-5690), known to regulate PTEN (Li Z et al. (2005) Nat Cell Biol 7, 399-404), and has been shown to be required for chemoattractant-induced recruitment of neutrophils and activation of cell-cycle component Cdc42 in the mouse (Li Z et al. (2003) Cell 114, 215-227). While an ARHGEF6 −/− has been created and shows no gross defect (Li Z et al. (2003) Cell 114, 215-227), no kidney ischemic-reperfusion injury phenotype has yet to be reported for this model. ARHFEG6 is currently known to have at least three missense single nucleotide polymorphisms, one of which has an average minor allele frequency as high as 0.43, suggesting there are large prevalent differences in the structure of this protein across populations.

We also show that 15 patients demonstrate an increase in antibodies against Stathmin-like 3 (STMN3, also known as SCUP), a protein we show is expressed in the renal pelvis. STMN3 has been previously independently shown to be expressed at a moderate level in cells from the human kidney (Bieche I et al. (2003) Genomics 81, 400-410). Pig models of kidney ischemic-reperfusion injury have shown that expression amounts of a related gene, STMN1, is correlated with reduction of ischemia (Jayle C et al. (2007) Am J Physiol Renal Physiol 292, F1082-1093). Mouse models of kidney ischemic-reperfusion injury have shown that STMN1 is increased in expression in renal tubule cells and is necessary for the recovery phase (Zahedi K et al. (2006) Am J Physiol Renal Physiol 290, F1559-1567). While a STMN3 −/− has been created (Lexicon Genetics), no kidney ischemic-reperfusion injury phenotype has yet to be shown for this model.

The presence, immunogenicity, and significance of pelvis epithelial antigens in human transplants have not been previously studied and the extent of pelvis specific allo-antibody responses may be dependent on the avidity of the antibody, and the dose of the antigen presented. The findings that the pelvis compartment of the kidney shows the greatest intensity of de novo post-transplant allo-antibodies, that ARHGEF6 and STMN3 are confirmed to localize in the renal pelvis, and both mount robust antibody responses after transplantation of the kidney, all enable us to consider a common mechanism that links all of these findings together: the role of peri-operative ischemia and reperfusion injury which may expose specific antigenic injury targets in the pelvis early after transplantation, and ongoing (and as yet unexplained) post-transplant injury triggers for the renal pelvis and parenchyma. Peri-operative factors (brain death, surgery, cold storage, reperfusion) are known to lead to ischemic injury in the renal pelvis, and in very rare but extreme conditions, have been shown to lead to even pelvic necrosis (Hidalgo G et al. (2000) Pediatr Transplant 4, 60-62). Reperfusion injury is known to lead to STMN1 up-regulation, and leads to neutrophil recruitment (Koo D D et al. (1998) Am J Pathol 153, 557-566.). During this process, it is plausible that ARHGEF6 protein is also up-regulated in these neutrophils. This speculative model could be tested in a mouse model with measures of specific protein increases in STMN3 and ARHGEF6, in combination with escalating doses of FK506 or other immunosuppressive agents, which are known to reduce peri-operative ischemia and reperfusion injury.

The outer cortex is also a critical source of allo-immunogenicity, as demonstrated in this study. This is not surprising as functionally significant injury in the kidney transplant is scored and recognized in the renal cortex (Racusen L C et al. (1999) Kidney Int 55, 713-723) and the glomerulus. Peri- and post-transplant triggers for cortical and glomerular injury include acute rejection, infection, hypertension, and pharmaceutical agents, including the immunosuppressive drugs used for maintenance therapy in these patients. It is speculated that these cumulative injuries may result in the recognition of compartment specific antigenic targets after transplantation, with generation of de novo non-HLA antibodies.

It is still not clear why these intracellular antigens are targeted by an allo-immune response; many of these responses being potentially against kidney alloantigens; these proteins are not known to be expressed at the cell surface. Our proposed allo-antigens here are not the first intracellular peptides seen as auto-antibodies (Jordan P & Kubler D (1995) Molecular biology reports 22, 63-66). One possibility is that immune exposure of these antigens happens secondarily to primary events such as tissue damage from ischemia or damage from reactive oxygen species after reperfusion, which could release normally intracellular peptides for immune presentation. A second possibility is that there are increased levels or unusual forms of these proteins in renal tubule, infiltrative neutrophils, and other cells in response to transplantation. A third possibility is that under conditions of physiological stress, proteins may be expressed and targeted to the cell surface (Jordan P & Kubler D (1995) Molecular biology reports 22, 63-66).

The next step in this study is to look at a targeted group of antibodies to these minor non-HLA targets and examine them for their correlation with clinical graft outcomes. As the samples examined in this study do not have clinical graft dysfunction categories, correlation of these antibodies with decline in renal graft function or graft survival could not be performed. Further studies are necessary to determine how these antibody levels, as measured by protein microarrays, correspond to clinical differences, particularly examining their impact on chronic graft injury, and how they change longitudinally. If clinically significant, these levels could be followed to titer pharmacological immunosuppression, or could be studied as a target for depletion. Additional work needs to be done to explain why these antibodies are formed, and whether DNA variants are present in the genes coding for these proteins between donor and recipient are present. The role of the renal pelvis as an immunogenic compartment needs to be explored, especially as a function of varying surgical and medical techniques to limit ischemia and reperfusion injury.

In summary, the utility of high-density protein microarrays to study post-transplantation responses is clear, and the techniques of integrative proteo-genomics can now be extended to this novel measurement modality to successfully and statistically filter measured responses to just those associated with a particular anatomical compartment. Putting together our high-density protein microarray data with publicly-available gene expression microarray data has yielded more than just the sum of the parts, and more specific questions and hypotheses to target in renal transplantation.

### Example 2

### Protein Microarrays Identify Antibodies to Protein Kinase Cc that are Associated with a Greater Risk of Allograft Loss in Pediatric Renal Transplant Recipients

Antibodies to human leukocyte antigens (HLAs) are a risk factor for acute renal allograft rejection and loss. The role of non-HLAs and their significance to allograft rejection have gained recent attention. Here, we applied protein microarray technology, with the capacity to simultaneously identify 5056 potential antigen targets, to assess non-HLA antibody formation in 15 pediatric renal transplant recipients during allograft rejection. Comparison of the pre- and post-transplant serum identified de novo antibodies to 229 non-HLA targets, 36 of which were present in multiple patients at allograft rejection. On the basis of its reactivity, protein kinase Cζ (PKCζ) was selected for confirmatory testing and clinical study. Immunohistochemical analysis found PKCζ both within the renal tissue and infiltrating lymphocytes at rejection. Patients who had an elevated anti-PKCζ titer developed rejection, which was significantly more likely to result in graft loss. The absence of C4d deposition in patients with high anti-PKCζ titers suggests that it is a marker of severe allograft injury rather than itself being pathogenic. Presumably, critical renal injury and inflammation associated with this rejection subtype lead to the immunological exposure of PKCζ with resultant antibody formation. Prospective assessment of serum anti-PKCζ levels at allograft rejection will be needed to confirm these results.

### Introduction

Although advances in allograft allocation and immunosuppression have reduced the incidence of acute rejection (AR) episodes after renal transplantation, AR remains a significant risk factor for allograft failure (Wissing K M et al. Transplantation 2008; 85: 411-416; Hariharan S et al. N Engl J Med 2000; 342: 605-612). Donor-specific antibodies (DSAs), are widely recognized as a risk factor, both for AR and for allograft loss (Mao Q et al. Am J Transplant 2007; 7: 864-871). Recently, antibodies to non-human leukocyte antigens (non-HLAs) have been the subject of more intense scrutiny. The Collaborative Transplant Study described 4048 HLA-identical sibling transplants (Opelz G. Lancet 2005; 365: 1570-1576). In the course of 10 posttransplant years, a higher panel-reactive antibody was associated with significantly lower allograft survival. As these transplants involved HLA-identical siblings, the increase in allograft loss could not be attributed to DSAs. This study did not specifically detect non-HLA antibodies nor did it show causality, but it clearly established the negative impact of ‘non-HLA immunity’ on allograft survival and function. Collins et al. (Collins A B et al. Transplant Proc 2006; 38: 3427-3429) described C4d deposition in the absence of DSAs in HLA-identical, ABO-compatible renal allograft recipients who had experienced allograft failure. Although they were unable to investigate or identify non-HLA antibodies in these patients, the occurrence of presumed antibody-mediated rejection in these HLA-identical patients was thought to be caused by non-HLA alloantibody production.

Thus far, only a few non-HLA antibodies have been identified in humans (Carter V et al. Transplant Proc 2005; 37: 654-657; Dragun D et al. N Engl J Med 2005; 352: 558-569; Zou Y et al. N Engl J Med 2007; 357: 1293-1300; Sun Q et al. Transplantation 2005; 79: 1759-1762; Sun Q et al. Clin J Am Soc Nephrol 2008; 3: 1479-1486). In addition, the absence of commercially available, validated detection strategies has hampered our ability to determine their clinical relevance and ascertain whether these antibodies are truly pathogenic (Dragun D. Transplantation 2008; 86: 1019-1025; Tinckam K J and Chandraker A. Clin J Am Soc Nephrol 2006; 1: 404-414). Protein microarrays offer a novel technique for the identification of patient-specific serum antibodies to non-HLA immunological targets, allowing simultaneous detection of antibodies to thousands of potential antigens. Although this technique has been applied to human autoimmune and oncological disease, our study represents the first use in the field of solid organ transplantation (Hudson M E et al. Proc Natl Acad Sci USA 2007; 104: 17494-17499; Robinson W H et al. Nat med 2002; 8: 295-301).

We applied protein microarray technology to 15 pediatric patients who had experienced AR after renal transplantation.

By paired comparative analysis using both pre-transplant and posttransplant serum samples (Li L et al. Proc Natl Acad Sci 2009; 106: 4148-4153), the protein microarray was able to identify 36 de novo antibody targets that were present in at least two patients at AR. In addition, a high antibody titer to one of these targets, protein kinase C-ζ0 (PKCζ), was associated with a recalcitrant subtype of AR and a significantly greater risk of allograft loss.

### Methods

Patient Selection

A review of our pediatric transplant database identified patients who had undergone renal allograft transplantation and experienced at least one episode of acute allograft rejection. A total of 15 patients were selected based on availability of serum samples, both before transplantation and at the time of AR. All transplant allograft biopsies were graded on the basis of the Banff classification (Racusen L C et al. Kidney Int 1999; 55: 713-723; Solez K et al. Am J Transplant 2008; 8: 753-760).

Pre-transplant serum samples were obtained within 48 h before allograft placement. The at-AR serum samples were obtained concurrently with the biopsy showing AR and before initiation of anti-rejection therapy. No patients received antibody therapy, including intravenous immunoglobulin, before the sample being obtained. Anti-HLA testing was performed as standard posttransplant care and the results were obtained from our histocompatibility laboratory. Pre- and posttransplant serum samples from all 15 AR patients were processed for ProtoArray and ELISA experiments. An additional 28 stable, posttransplant pediatric renal allograft recipients were selected as controls for the ELISA analysis using our validated PKCζ ELISA. These 28 patients were chosen based on clinical similarity to the 15 AR patients and the presence of a posttransplant surveillance biopsy showing the absence of AR. Serum samples for these patients were obtained concurrently with the biopsy showing the absence of AR. Pre-transplant serum samples were not available for these 28 allograft recipients. These serum samples were processed for ELISA experiments. All serum samples were available under a previously institutional review board approved protocol (no 13443).

Identification of Autoantibody Targets Using Protein Microarray

A total of 30 protein microarrays (ProtoArray V3; Invitrogen, Carlsbad, Calif., USA) were used for this study, one each for the pre-transplant and the at-AR serum samples of the 15 patients with AR. The ProtoArrays were blocked with blocking buffer for 1 h followed by application of plasma sample (1:150) for 90 min. After washing the protein microarray four times for 10 min each, the protein microarrays were probed with secondary antibody (goat anti-human Alexa 647, Molecular Probes, Eugene, Oreg., USA) for 90 min. After washing the slides, the protein microarrays were dried and scanned using a fluorescent microarray scanner (GSI Luminoics, Perkin-Elmer scanner, Waltham, Mass., USA). All steps were carried out on a rotating platform and at 4° C. The slides were scanned at a photomultiplier gain of 60% with a laser power of 90% and a focus point of 0 gm. The ‘.gal’ files were obtained from a ProtoArray central portal on the Invitrogen website (www.invitrogen.com/ProtoArray) by submitting the barcode of each protein microarray. Data was obtained using GenePix software (Version 6, Molecular Devices, Sunnyvale, Calif., USA). Using the appropriate ‘.gal’ file and the respective microarray image obtained from the scanners. Novel alloimmune antibody responses are identified by subtracting the pre-transplant data set from the posttransplant data set (delta); all reported ProtoArray signal intensities represent the delta intensity (signal at AR-signal pre-transplant). A target response was considered positive, and indicative of de novo antibody formation, if the response delta, defined as the response intensity at AR subtracting the pre-transplant response intensity, was arbitrarily 500 or greater. Positive antibody responses were arranged according to occurrence frequency, and all targets identified in at least two patients were reviewed with specific attention directed at the strength of the antibody response, human tissue expression data, gene ontology of the target, and the relevance to immunological function. Given the preliminary nature of this study, a single target, PKCζ C, was selected as a candidate target for further analysis on the basis of the aforementioned factors.

ELISA Validation of PKCζ Protein Microarray Results

Both the pre-transplant and the at-AR serum samples from the 15 AR patients and the posttransplant serum samples from the 28 stable kidney transplant recipients were analyzed by ELISA. Insect cell-expressed human recombinant protein, PKCζ C was obtained from Invitrogen. The 96-well microwell ELISA plate was coated with 0.27 pg PKCζ C protein in 50 μl coating buffer (15 mM Na₂CO₃, 30 mM NaHCO₃, 0.02% NaN₃, pH 9.6) and incubating overnight at 4° C. The standard curve was generated using rabbit polyclonal antibody to PKCζ C (Abcam, Cambridge, Mass., USA), and Zymax-grade AP-conjugated goat anti-rabbit IgG (Invitrogen). After washing the plate with tris-buffered saline tween 20 buffer five times, the non-specific protein binding was blocked by 100 μl, 2% dry milk in tris-buffered saline tween 20 buffer for 1 h at room temperature. After the blocking step, 50 μl serum samples (40-fold diluted with 2% milk in tris-buffered saline tween 20 buffer) were incubated on the wells for 1 h at room temperature. The plate was washed five times with tris-buffered saline tween 20 buffer and incubated in 50 μl AP-conjugated AffiniPure Mouse anti-human IgG (Jackson ImmunoResearch, West Grove, Pa., USA). The color was developed by using AP-pNPP liquid substrate system for ELISA (Sigma-Aldrich, St Louis, Mo., USA). Absorption was measured at 405 nm with a SPECTRAMax 190 microplate reader (Molecular Devices, Sunnyvale, Calif., USA). Serum PKCζ C antibody concentrations were determined from the standard curve.

Longitudinal Allograft Survival Analysis

Allograft survival was assessed in the 15 patients with AR in the study set. Patients were divided into AR subtypes based on their serum anti-PKCζ C levels at AR: 3 with high serum anti-PKCζ C levels and 12 with low serum anti-PKCζ C levels. Follow-up commenced at the time of the initial AR event. Follow-up was continued until allograft loss occurred or until the time of most recent assessment of allograft function. Allograft loss was defined as a return to dialysis.

IHC Staining for PKCζ in Renal Parenchyma

Immunohistochemical staining was performed using antibodies directed against PKCζ C (GeneTex, San Antonio, Tex., USA catalog no GTX40214). Formalin-fixed, paraffin-embedded tissue were pretreated with citrate and stained with polyclonal antiserum to PKCζ C (dilution 1:2000 for 18 h). A rabbit ABC detection kit (Vector Labs, Burlingame, Calif., USA) was used (PK-6101). Negative controls were run to assess for non-specific anti-PKCζ C staining.

Statistical Analysis

t-Test, ANOVA (analysis of variance), and χ²-test were used for analysis of continuous or categorical types of data. Correlation analysis was performed for antigens detected by ProtoArray and ELISA. Graft survival rate was based on Kaplan-Meier survival analysis at current follow-up. P-values ≦0.05 were considered statistically significant. Results are reported as mean±standard deviation. All statistical analyses were performed using SAS 9.1.3 (SAS Institute, Cary, N.C., USA).

Results

Antigen Discovery Using Protein Microarray

A total of 15 pediatric (mean age at transplantation 12.4±5.2 years) kidney transplant patients, with a mean HLA mismatch score of 4.1, were examined in our antigen discovery phase (FIG. 9). In total, 12 patients received steroid-free maintenance immunosuppression consisting of tacrolimus and mycophenolate mofetil, whereas the three remaining patients received steroid-based maintenance immunosuppression consisting of tacrolimus, mycophenolate mofetil, and prednisone. The patients developed AR at a mean of 22.3±20.7 months posttransplant. All patients experienced acute cellular rejection of whom four patients had Banff 1a rejection, eight patients had Banff 1b rejection, and three patients had Banff 2a rejection. Of the 15 patients with cellular rejection, only 4 (27%) had additional evidence of antibody-mediated rejection, based on positive C4d staining and the presence of DSAs. However, 53% (8/15) had at least one DSA at AR, whereas an additional 27% (4/15) had at least one non-DSA HLA antibody detected at AR.

At AR, de novo, serological, non-HLA responses were detected against 4.5% of the protein microarray targets (229/5056). At least one target was recognized in all patients, 36 targets were identified in at least two patients at AR. The mean protein microarray delta signal intensity of these targets in their respective patients was 1390±1061 intensity units compared with the mean delta signal intensity for all 5056 targets across all of the 15 patients, which was 7.6±198.3 (standard error, 0.7) intensity units. Patients with detectable anti-HLA recognized a mean of 24.4±15.4 non-HLA antigen targets. Patients without evidence of anti-HLA recognized a mean of 79.3±108.9 non-HLA antigen targets. This difference was not statistically significant (P=0.47); the greater mean number and larger standard deviation of non-HLA antigen targets recognized in patients without anti-HLA reactivity was primarily due to the fact that one of the three patients in this group recognized substantially more non-HLA antigens (205).

As this was a pilot study designed to assess the utility of the protein microarray technique in pediatric renal transplant recipients, we chose to focus our analysis on a single target, PKCζ, which had the highest mean signal intensity (6408 intensity units) of all 36 targets that were identified in two or more patients. In addition to having the strongest mean ProtoArray (Invitrogen, Carlsbad, Calif., USA) signal, PKCζ was known to be expressed within renal parenchymal tissue, and has been shown to be actively involved in regulation of inflammation, cell survival, and apoptosis (Leitges M et al. Mol Cell 2001; 8: 771-780; Leroy I et al. Cell Signal 2005; 17: 1149-1157; Leseux L et al. Blood 2008; 111: 285-291; Padanilam B J. Kidney Int 2001; 59: 1789-1797; San-Antonio B et al. J Biol Chem 2002; 277: 27073-27080; Xin M et al. J Biol Chem 2007; 282: 21268-21277; Zhao Y et al. J Invest Dermatol 2008; 128: 2190-2197; Huang X et al. J Immunol 2009; 182: 5810-5815; Chen C et al. J Surg Res 2009; 153: 156-161).

Antigen Validation of Protein Microarray Results by Enzyme-Linked Immunosorbent Assay

Protein kinase C-ζ was analyzed by enzyme-linked immunosorbent assay (ELISA) across all 15 AR patients of the study set; ELISA showed a significant positive correlation with the protein microarray results (R²=0.84, P-value <0.001). Confirmation of ProtoArray-detected antibody presence and signal intensity, to our knowledge, has been validated for the first time in this study by ELISA. ELISA-determined at-event serum anti-PKCζ levels were plotted for the pre-transplant and the at-AR samples for each of the 15 patients, as well as for the posttransplant samples of 28 stable posttransplant patients who served as controls (FIG. 10). The clinical characteristics of these control patients were similar to those of the 15 patients experiencing AR, with the exception of event time posttransplant (FIG. 9). AR occurred, on average, 22.3±20.7 months after transplant, whereas the biopsy showing the absence of AR occurred, on average, 6.6±3.4 months after transplant in the control patients (P<0.005). The mean anti-PKCζ serum levels for the pre-transplant, at-AR, and posttransplant stable control samples were 30.9±5.1 pg/μl, 46.7±34.9 pg/μl, and 34.8±8.6 pg/μl, respectively. Although there was a slight trend toward higher anti-PKCζ levels in the at-AR samples, this failed to reach statistical significance (P=0.07).

When the at-AR samples were further analyzed, the anti-PKCζ levels determined by ELISA were dramatically higher in 3 of the 15 AR patients, who all had values >75 pg/μl (FIG. 10). The mean anti-PKCζ level in these three patients was 109±34.4 pg/μl. This was significantly greater than the mean anti-PKCζ level in the remaining 12 patients, 31.1±3.1 pg/μl (P<0.001). Comparative HLA and biopsy information for the patients with high anti-PKCζ levels and low anti-PKCζ levels is shown in FIG. 11. There was no association between the presence of high anti-PKCζ levels and the pathological severity of rejection, as graded by the Banff criteria (P=0.63), or a diagnosis concurrent to antibody-mediated rejection (P=0.24). In addition, there was no correlation between high anti-PKCζ levels and the presence of dense CD20-positive cell clusters (P=0.44). Finally, there was no association between high anti-PKCζ titers and development of antibodies to HLA targets. This held true both for DSA (P=0.60) and non-DSA HLA antibodies (P=0.44).

Allograft Survival Analysis

When the high anti-PKCζ and the low anti-PKCζ patients were assessed by Kaplan-Meier analysis (FIG. 12), at a mean follow-up of 4.5±0.5 years, the low anti-PKCζ patients had significantly better allograft survival than the patients with high anti-PKCζ levels (100% versus 33%; P=0.002). Although 4 of the 15 AR patients had C4d staining evident in their AR biopsy, none of the three patients with high anti-PKCζ levels had positive C4d staining.

Immunohistochemical Staining for PKCζ

To evaluate the localization of the PKCζ antigen in the transplant and the native kidney, immunohistochemical (IHC) staining was performed. PKCζ was shown in native and transplanted, non-rejecting kidney tissue, localizing both to the smooth muscle layer of arterioles and to the cytoplasmic domain of distal tubular cells (FIGS. 13a and b). IHC staining of renal allografts during AR shows the presence of PKCζ additionally in lymphocytes, both within lymphocyte aggregates and scattered throughout the tubulointerstitium (FIGS. 13c and 13d).

Discussion

These results show the feasibility of applying protein microarrays to renal transplant recipients. It is a novel and emerging technology with the capacity to identify thousands of potential immunogenic non-HLA antigens. Previously, Robinson et al. (Robinson et al. Nat med 2002; 8: 295-301) fabricated an 1152-feature protein micro-array that was used to show specific autoantibody binding and characterize sera in known autoimmune disease states. The currently used ProtoArray platform from Invitrogen offers a human protein microarray containing 5056 antigens. To date, there is a single publication using this technology to study human disease. In this study, the ProtoArray was probed with sera from patients with ovarian cancer (Hudson M E et al. Proc Natl Acad Sci USA 2007; 104: 17494-17499). Although no target was identified universally in patients with ovarian cancer, autoantibodies to four antigens were found to have higher reactivity in patients with ovarian cancer when compared with healthy controls. Although not all patients with ovarian cancer formed detectable auto-antibodies to these targets, combined immunostaining for two of the targets identified by protein microarray led to a highly sensitive and specific tissue diagnosis tool.

We have used the ProtoArray for the first time in solid organ transplantation to determine whether de novo, non-HLA targets, with clinical and prognostic relevance, can be identified in transplant patients experiencing AR. With this technology, we found biologically relevant antibody targets in multiple patients at AR. Interestingly, the repertoire of antigens recognized seems to be patient specific, with variable reactivity to the range of protein targets; the patients had antibody responses to between 0.1% and 4.1% of the possible antigens. In addition, in our small cohort, the number and specificity of antigen targets recognized did not seem to be associated with the development of HLA antibodies. In total, 36 of the 5056 antigens were recognized in at least 2 of the 15 AR patients. This seemingly low number is not surprising given that the ProtoArray was not designed to examine renal-related antigens or transplant-specific targets. It is likely that a protein microarray optimized for solid organ transplantation would have a higher net yield. Despite this, we were able to identify antibodies to numerous biologically relevant antigen targets, simultaneously using a single test and minimal patient serum. Given the preliminary nature of this study, we chose one such relevant target, PKCζ for additional analysis. PKCζ was chosen because it had the strongest mean signal intensity of the 36 potential antigens, it is known to be present in renal tissue, and it is involved in inflammatory signal transduction pathways. Comprehensive analysis of other relevant targets will be the focus of future investigation.

Protein Kinase C-ζ which is expressed in a number of tissues, including brain, kidney, lung, and testes (SOURCE Search for PRKCZ. http://smd.stanford.edu/cgi-bin/source/sourceResult. Accessed on June 2008), is an atypical PKCζ which is an integral component of several pathways involved in cell survival, proliferation, and apoptosis (Leroy I et al. Cell Signal 2005; 17: 1149-1157; San-Antonio B et al. J Biol Chem 2002; 277: 27073-27080; Xin M et al. J Biol Chem 2007; 282: 21268-21277). Animal model data are concordant with available in vitro data, suggesting that PKCζ has an active, regulatory role in inflammation. PKCζ deficient mice (PKCζ−) have reduced Peyer's patch formation, a relative reduction of B cells in peripheral lymph nodes, and no B-cell follicle formation (Leitges M et al. Mol Cell 2001; 8: 771-780). In addition, they lack the anti-apoptotic signal mediated by tumor necrosis factor-α-activated NF-kB, which is present in normal mice. In a renal ischemia/reperfusion rat model, PKCζ had significantly upregulated expression during the first hour of reperfusion, at 1 day after reperfusion, and at days 5-7 after reperfusion (Padanilam B J. Kidney Int 2001; 59: 1789-1797). Human studies have been consistent with the in vitro and animal model data, establishing the active role PKCζ C has in inflammatory cell signaling and cell survival. PKCζ C is involved in intracellular signaling in human monocytes and macrophages, and mediates lipopolysaccharide-activated pro-inflammatory cytokine gene expression (Huang X et al. J Immunol 2009; 182: 5810-5815). In addition, PKCζ C mediates regulation of the mitogen-activated protein kinase and mammalian target of rapamycin pathways in follicular lymphoma cells, and seems to exert a survival function in these cells. Administration of rituximab, a humanized ant-CD20 immunotherapy, led to reduced PKCζ C activity and inhibited its survival effects (Leseux L et al. Blood 2008; 111: 285-291). Finally, Zhao et al (Zhao Y et al. J Invest Dermatol 2008; 128: 2190-2197) recently showed increased PKCζ C expression in psoriatic skin lesions compared with healthy skin. Tumor necrosis factor-α, a well-described pathogenic factor in psoriasis, was found to be dependent on PKCζ for cell signaling and signal transduction. After tumor necrosis factor-α stimulation, cytoplasmic and nuclear staining for PKCζ was increased. Furthermore, activation of PKCζ C was associated with an increased expression of CD1d, which interacts with natural killer T cells, and has an integral role in their cytokine production. Thus, PKCζ C seems to have a significant role in inflammatory cell signaling and may be upregulated in inflammatory disease states, such as acute allograft rejection.

In our analysis, although there was a slight trend toward higher anti-PKCζ C levels in the at-AR cohort compared with the pre-transplant and stable posttransplant cohorts, this trend failed to reach statistical significance. However, a subset of patients within the AR cohort had robust anti-PKCζ C responses, suggesting the presence of an AR subtype. When allograft survival was assessed, the patients with elevated anti-PKCζ C levels had significantly worse outcomes and anti-PKCζ C levels were significantly associated with accelerated allograft loss at mean follow-up of 4.5±0.5 years.

It is important to interpret these results with caution; given the small size of our cohort, we cannot rule out that anti-PKCζ C levels are elevated merely because of increased expression, abnormal splicing or protein folding, or polymorphism. In addition, although high anti-PKCζ C titers were significantly associated with allograft loss in our study, there is no evidence of causality. In fact, given that none of the three patients with high anti-PKCζ C titers had evidence of C4d deposition in their AR biopsies, it is likely that anti-PKCζ is a marker, or bystander molecule, related to cellular damage associated with severe AR, rather than being truly pathogenic. The fact that higher anti-PKCζ C levels were not associated with development of HLA antibodies would also suggest a different mechanism than that seen with DSAs in antibody-mediated rejection. Our IHC results show that PKCζ C is indeed present in renal parenchymal cells, localizing to smooth muscle and distal tubular cells in healthy renal allograft tissue. The presence of PKCζ C within renal tubular cells is consistent with a recent study which showed that PKCζ C is present in and regulates organic anion transporters in renal proximal tubular cells (Barros S A et al. J Biol Chem 2009; 284: 2672-2679). Interestingly, in the setting of AR, our IHC staining also found PKCζ within infiltrating lymphocytes, suggesting either upregulation within the inflammatory cell or immunological exposure to the intracellular antigen. Our results are consistent with the premise that PKCζ is upregulated in the inflammation associated with AR; we hypothesize that in our subset of AR patients with high anti-PKCζ levels, severe renal injury and cell death led to immunological exposure of PKCζ with resultant antibody formation. In this setting, the elevated anti-PKCζ titer may be a marker for the damage associated with a more severe subtype of AR. Interestingly, in our small pilot study, there did not seem to be a specific histological feature that was associated with AR and the presence of higher anti-PKC levels; however, it is possible that in a larger patient cohort such a characteristic might be found.

In summary, protein microarrays were able to successfully identify AR-specific antigenic targets in a high throughput manner and represent an appealing technology to better assess alloimmunity in solid organ transplantation. In addition, based on our results, PKCζ is a potential non-HLA antigen target recognized in pediatric renal transplant patients experiencing AR. It is not a target in all AR episodes, but there seems to be a subtype of AR, characterized by exposure of and antibody formation against PKCC, which is associated with poor allograft survival. Our results suggest that anti-PKCζ is a marker, rather than a truly pathogenic antibody and further research is necessary to accurately define the role that PKCζ has in AR.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of diagnosing or predicting graft status or outcome comprising:
a. providing a sample from a subject who has received an allograft;
b. determining the presence or absence of a protein recognizing a non-HLA/non ABO antigen, wherein said non-HLA/non ABO antigen is an allograft-specific antigen; and
c. diagnosing or predicting graft status or outcome based on the presence or absence of said protein.

2. The method of claim 1 wherein said graft status or outcome comprises acute rejection, chronic rejection, tolerance, non-rejection based allograft injury, graft function, graft survival, chronic graft injury, or titer pharmacological immunosuppression.

3. The method of claim 2 wherein said non-rejection based allograft injury is selected from the group of ischemic injury, virus infection, peri-operative ischemia, reperfusion injury, hypertension, physiological stress, injuries due to reactive oxygen species and injuries caused by pharmaceutical agents.

4. The method of claim 1 wherein said sample is selected from the group consisting of blood, serum, urine, and stool.

5. The method of claim 1 wherein said non-HLA/non ABO antigen is an allograft-compartment specific antigen.

6. The method of claim 1 wherein said allograft is selected from the group consisting of kidney allograft, heart allograft, liver allograft, pancreas allograft, lung transplant, intestine transplant and skin allograft.

7. The method of claim 5, wherein said allograft is a kidney allograft and said compartment is selected from the group consisting of glomerulus, renal pelvis, outer cortex, inner cortex, outer medulla, inner medulla, and papilliary tip.

8. The method of claim 1 wherein said protein is an antibody.

9. The method of claim 1 wherein said non-HLA/non ABO antigen is selected from the group consisting of ARHGEF6, PPFIBP2, NIF3L1, ANXA10, STMN3, FAH, SLC6A6, CISD1, CYP4F11, PEX7, PECI, PMM1, IYD, CTNND1, CLIC2, PARVA, CMAH, FOXI1, MFI2, HSPA2, CLDN1, HCFC1R1, MYL4, MPZL2, AFAP1L2, GMPR, MGAT4B, OCLN, MFI2, TMEM61, and PKCζ.

10. The method of claim 9 wherein said non-HLA/non ABO antigen is selected from the group consisting of ARHGEF6 and STMN3.

11. The method of claim 9 where said non-HLA/non ABO antigen is PKCζ.

12. The method of claim 1 further comprising determining the presence or absence of a plurality of proteins recognizing non-HLA/non ABO antigens, wherein said non-HLA/non ABO antigens are allograft specific antigens.

13. The method of claim 12, wherein said non-HLA/non ABO antigens are allograft-compartment specific antigens.

14. The method of claim 13 wherein said proteins are antibodies.

15. The method of claim 12 wherein the presence or absence of said plurality of proteins is determined with a protein microarray.

16. The method of claim 13 further comprising measuring the expression of said non-HLA/non ABO antigens.

17. The method of claim 16 wherein said measuring the expression of said non-HLA/non ABO antigens comprises PCR or microarrays.

18. The method of claim 1 wherein said subject has received immunosuppression therapy.

19-31. (canceled)

32. The method of claim 14 wherein said method has at least 56% sensitivity.

33. (canceled)

34. (canceled)

35. The method of claim 14 wherein said method has a specificity of about 80% to about 100%.

36-73. (canceled)
